(19) United States
(12) Patent Application Publication
(10) Pub. No.: US 2011/0048427 A1
(43) Pub. Date: Mar. 3, 2011

(54) METHOD AND APPARATUS FOR BLOCKING FLUID FLOW IN AN INTUBATED TRACHEA

(76) Inventor: Oron Zachar, Tel Aviv (IL)

(21) Appl. No.: 12/823,114

(22) Filed: Jun. 24, 2010

Related U.S. Application Data
(60) Provisional application No. 61/350,913, filed on Jun. 2, 2010.
(60) Provisional application No. 61/329,106, filed on Apr. 29, 2010.
(60) Provisional application No. 61/238,151, filed on Aug. 29, 2009.
(60) Provisional application No. 61/219,769, filed on Jun. 24, 2009.
(60) Provisional application No. 61/236,553, filed on Aug. 25, 2009.

(30) Foreign Application Priority Data
Oct. 27, 2009 (US) PCT/US2009/062227
Jun. 23, 2010 (GB) GB 1010564.1

(51) Int. Cl. A61M 16/04

(52) U.S. Cl. 128/207.15

(57) ABSTRACT

Disclosed are systems, kits and methods for facilitating intubation of a patient. In some embodiments, a system includes an ETT tube that longitudinally traverses the interior of a sleeve. Preferably, the sleeve is outwardly biased, includes a fibrous skeleton, and is covered by an elastic, substantially-liquid-impermeable coating. Some embodiments relate to an apparatus technique for preventing downward movement of liquid by a connecting membrane Some embodiments relate to kits which, when assembled, provide any ETT system disclosed herein. Some embodiments relates to method of assembling, method of deploying, and methods of removing the ETT system or a portion thereof.

### RELATED APPLICATION INFORMATION

This application claims priority from U.S. Provisional Application Ser. Nos. 61/219,769, filed on Jun. 24, 2009; 61/236,553, filed on Aug. 25, 2009; 61/238,151, filed on Aug. 29, 2009; 61/329,106 filed on Apr. 29, 2010; 61/350,913 filed on Jun. 2, 2010. This application also claims priority to PCT/US2009/062227 filed on Oct. 27, 2009. This application also claims priority to British Patent Application Serial Number GB 1010564.1 filed on Jun. 23, 2010. The contents of all of these previously-filed patent applications are incorporated herein by reference in their entirety.

### FIELD OF THE INVENTION

Embodiments of the present invention relate to an apparatus, method and kit for reducing a likelihood of fluids leaking from the trachea of a patient undergoing intubation into a lung thereof.

### BACKGROUND AND RELATED ART

During administration of anesthesia to a patient, or in situations in which the patient is in the intensive care unit (ICU), it is standard practice to intubate the patient with a tube, introduced into the trachea, to facilitate pulmonary ventilation. The present state of the art of intubation is illustrated in FIG. 1A, which is taken from FIG. 4A of U.S. Pat. No. 6,843,250. As illustrated in FIG. 1A, an endotracheal tube (ETT) 100 which is inserted into a patient airway 111, typically includes an inflatable balloon or cuff 112 near its distal end. The cuff 112, when inflated, performs a dual function: (a) the cuff occludes the air passageway, thus establishing a closed system whereby the gas pressure in the airway distal to the inflated cuff can be maintained at a desired level, thus providing means of controlling the exchange of blood gasses in lungs. (b) the cuff provides a barrier against inflow of aspirated gastric contents or other matter foreign to the lungs.

Unfortunately, there are several complications often associated with such intubation. First, 8-28% of intubated patients develop ventilation associated pneumonia (VAP); there is 20-30% mortality rate among VAP patients. It is estimated that in the USA, VAP and associated complications increase patient time in intensive care units by about 4-6 days, at a cost increase of $20,000 to $40,000. VAP occurs because the insertion of the ETT bypasses the protective system of the tracheo-bronchial tree. Secretions, mucous or aspirated gastric material, which normally would be directed harmlessly through the digestive system, follow the path of the tube into the airways. Although the use of the balloon or cuff is supposed to prevent such fluids from entering the lungs, the cuff is not infallible. Inter alia, the inflatable cuff, on a typical commercially available endotracheal tube, is in the form of an oval-shaped balloon. The oval-shaped balloon permits these secretions to pool around the surface of the balloon proximal to the oral cavity, particularly in the vicinity of the region where the balloon contacts the tracheal wall; sometimes these fluids pass by the balloon and into the tracheo-bronchial tree. This passage of unwanted fluids past the inflated cuff of the tracheal tube device is thought to be due to the patient's breathing cycle producing fluctuating inhalation/exhalation pressures on the downstream ovate surface of the inflated cuff, causing the cuff and/or the tracheal conduit to act somewhat in the manner of a peristaltic pump.

FIGS. 1B-1D, all taken from previous patent documents, illustrate various conventional ETTs known in the art. The example of FIG. 1C includes a conventional cuff assembly mechanically coupled to the ETT. In FIGS. 1B-1D, it is possible to observe a tapered distal end—see, for example, element 17 of FIG. 1B. The example of FIG. 1B includes a so-called ‘Murphey eye’ near the distal end (see element 22 of FIG. 1B).

One salient feature of ETT tubes is that when in use they may be connected to a ventilation device (for example, via a collared gas connector 262 at proximal end of ETT as illustrated in FIG. 1C)—the ventilation device may provide gas flow in a distal direction into the human patient according to flow rates and/or pressure parameters known in the art—for example, according to FDA guidelines.

While it is standard protocol to attempt to suction the region in which fluid tends to collect, suctioning is awkward and, done blindly, may result in the insufficient suctioning of the pharynx. In advanced tube designs, the tube device is provided not only with an inflation line to the cuff but also with a suction line opening to a region above the cuff. In practice however, due to the finite axial length accommodated by the tape or other fastening means required to attach the cuff sealingly to the main tube of the structure, the opening from the suction line is disposed too far above the upstream ovate surface of the cuff to ensure removal by suction of all the unwanted fluids collecting in that region. Moreover, the oval shape of the cuff inevitably leads to having the most crucial area of fluid collection, at the contact between the cuff and the trachea surface, being too narrow for the reach of any suction device. Hence, suction above oval balloons may not ensure complete removal of all secretions.

Furthermore, when the cuff is deflated for removal, fluid collected on the upper surface of the cuff, proximal to the oral cavity, may flow into the lungs.

We believe the state of the art with regard to presently-used cuffs and the associated suction devices is represented by U.S. Pat. Nos. 5,259,371, 5,638,813, 7,089,942, 3,964,488, 4,979,505, 5,520,175, 5,937,861, 6,062,223, 7,089,942, and 7293561 and US patent publications nos. 20030024534 and 20080115789, and references therein. These patents and patent publications, as well as all other publications mentioned herein, are incorporated herein by reference.

A second major set of complications arising from tracheal intubation is associated with the cuff sealing pressure. To prevent the leakage of air from between the inflated cuff and the tracheal wall during mechanical ventilation, the pressure in the cuff must be equal to or greater than the peak inspiratory pressure within the airway. Peak inspiratory pressures are only achieved for 10%-25% of the ventilatory cycle but may be as high as 50 mm of mercury. Since the pressure within the standard cuff is static, to achieve continuous good sealing the cuff pressure must ideally be maintained at this relatively high pressure (equal to or greater than peak airway pressure) throughout the ventilatory cycle, to prevent leaks during the highest pressure portion of the cycle. Yet such sealing pressure cannot be implemented in practice due to the risk of tissue anoxia and other complications: as the cuff pressure exceeds the capillary pressure of the tracheal tissues (which is normally 25 mm of mercury), tissue anoxia occurs, and varying degrees of tracheal injury result. The injuries range from mild erosion of the mucosa, to destruction of the tracheal cartilage rings, to segmental tracheomalacia with dilatation of the trachea. More dramatic is full thickness erosion, with perforation of the inominate artery anteriorly or posteriorly into the esophagus; both of these events are associated with a high rate of mortality. Late complications of tracheal stenosis, from mild to incapacitating obstruction, are most often observed in patients requiring long-term ventilatory support, such as patients hospitalized in the ICU.

As illustrated in FIG. 1A, cuffed endo-tracheal tubes (cuffed ETT) currently in use employ a soft inflatable cuff balloon 112 that, when inflated within the trachea, assumes a fusiform shape presenting a surface in contact with the trachea mucosa. The cuff balloon 112 is inflatable from the outside the body via an inflating lumen 116. Any prolonged pressure above 25 torr increases the risk of tracheal necrosis. The state of the art in dealing with excess cuff pressure is described in U.S. Pat. No. 5,937,861. In some high end present art ETTs, there is an integrated suction lumen 122 with a distal port opening above the cuff balloon 112 and connected at the other end to an outside suction device 126, by which fluids collecting above the balloon can be sucked out. Therefore, conventional cuffed ETT devices with integral suction include three lumens: the ETT breathing tube 100, the cuff inflating tube 112, and the suction tube 126.

One of the contributing factors to the development of VAP in patient intubated with ETT is the blocking of fluids cleared upward by the cilia lining the trachea. In normal healthy people, fluids and contaminations (including particles and bacteria) are continuously cleared out from the lungs by cilia lining the tubes into the lungs, and are further pushed upward in the trachea towards the vocal cords, and eventually cleared out by cough. The cuff of conventional ETT may hinder activity of the cilia mid-way through the trachea so that contaminated fluids collect and drip back into the lungs.

In addition to endotracheal tubes, tracheal stents are known in the art. Unlike ETT's, however, which are generally used to control a patient's breathing, tracheal stents are merely used to keep the air passage open. The state of the art of tracheal stents is exemplified in www.emedicine.com/ent/topic593.htm and in Ann. Thorac. Med. (2006) 1:92-7, US patent publication 20030024534, PCT patent publication WO 2004/067060 A2, and references therein. The general art of stent construction and design is further exemplified in U.S. Pat. Nos. 7,291,166, 7,300,459, 7,374,570, 7,285,132, 6,821,291, 6,458,152, and 5,716,410, US patent publication no. 20080077222, and references therein. All of these documents are incorporated by reference in their entirety.

Of particular relevance to the present invention is the art of coated mesh stents. As illustrated in FIG. 2, such mesh stents may are commonly madge of linked segments or braided fibers 222, where the braid may be of single or multiple fibers. The coating 224 is commonly made of an elastic material such as silicon or polyurethane. The prior art of such stents is exemplified in U.S. Pat. Nos. 5,061,275, 5,158,545, 5,591,226, 6,162,244, 7,594,928 which are all incorporated by reference in their entirety. One salient feature of such stents is that they have the tendency to elastically self expand to their rest diameter if pre-compressed to a lesser diameter. Thus, in common delivery procedure of such stents, the stent is delivered pre-compressed to a lower diameter and then release to expand within a target body lumen such as a trachea.

Various techniques or systems have been proposed for retrieving and/or repositioning an implanted stent, as summarized within US patent application 20060276887 incorporated by reference in its entirety. For example, U.S. Pat. No. 6,676,692 to Rabkin et al. (incorporated by reference in its entirety) describes a catheter system having stent-capturing hooks. The hooks are described as being useful for engaging the stent, thereby allowing repositioning and/or retrieval of the stent.

U.S. Patent Application Publication No. 2002/0188344 A1 to Bolea et al, incorporated by reference in its entirety, describes the use of hinged hooks attached to interior portions of an implantable stent.

### SUMMARY OF EMBODIMENTS

Embodiments of the present invention relate to an apparatus, method and kit for reducing a likelihood of fluids leaking from the trachea of a patient undergoing intubation into a lung thereof.

Some embodiments provide a cuff assembly and a related method for preventing and/or hindering a downward motion of fluids in the trachea during intubation. The cuff assembly includes an elastic, outwardly biased, liquid-impermeable elongated sleeve (preferably constructed from a fibrous skeleton coated with a biocompatible elastic material) which, when deployed within the trachea, outwardly presses against the wall of the trachea.

The cuff assembly further includes a liquid-impermeable connecting element that is permanently attached to an inner surface of the sleeve. When an ETT passes through an inner region of the elongated sleeve to longitudinally traverse the sleeve, the connecting element (for example, comprising one or more liquid-impermeable non-rigid membranes) is in contact with both an outer surface of the ETT and an inner surface of the sleeve. This contact may provide a seal, in the interstitial area between the wall of the trachea and the outer surface of the tube, between a proximal portion of the human trachea above the cuff and a distal portion of the human trachea below the cuff.

In contrast to conventional ‘inflation-based’ cuff systems which generally rely on inflation to provide outward pressure on the trachea, embodiments of the present invention may employ an outwardly-biased sleeve constructed from a fibrous skeleton which is coated with a biocompatible elastic material (e.g. silicone, polyurethane or latex). Furthermore, the presence of the connecting element (e.g. an array of membranes) which radially spans the region within the inner surface of the sleeve and outside of the ETT to connect the sleeve to the ETT may be useful for ‘sealing’ an upper region above the connecting element from a lower region below the connecting element, to protect the lungs from downward motion of liquids into the lungs.

Some embodiments of the present invention relate to kits including the ETT and the cuff assembly, and to methods of assembling the same.

Some embodiments of the present invention relate to kits and/or apparatus for loading the cuff assembly and ETT into the patient's trachea, and to methods of accomplishing the same.

Some embodiments of the present invention relate to kits and/or apparatus for removing the cuff assembly and ETT from the patient's trachea, and to methods of accomplishing the same.

Some embodiments of the present invention relate to an improved ETT, for example, including one or more flexible sections—for example, ‘accordion-like’ sections.

Some embodiments of the present invention relate to techniques and apparatus for providing suction to remove fluids from one or more locations in the trachea. In one non-limiting example, the suction device may include a port below the sealing, connecting element.

These and further embodiments will be apparent from the detailed description and examples that follow.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Embodiments of the invention will be explained below in greater detail with reference to the accompanying drawings, in which:

FIGS. 1A-1D illustrates a typical design and employment of an endotracheal tube (ETT), as known in the art;

FIG. 2 illustrates the prior art of mesh stents which may be coated with an impermeable elastic coating layer;

FIG. 3 illustrates the core structure of a sleeved ETT according to some embodiments;

FIGS. 4A-4E illustrate some examples of an ETT apparatus including an expandable sleeve and a connecting element deployed within a trachea;

FIGS. 5A-5B describe some exemplary geometric parameters;

FIG. 6 illustrates various aspects of an ETT system according to some embodiments;

FIG. 7-10 illustrate apparatus and techniques related to a sleeve loading/closing mechanism according to some embodiments.

FIG. 11A-11C illustrates an embodiment including a distal pulling element implemented as an array of wires that connect the distal end of the sleeve to the ETT.

FIG. 12 illustrates a technique for assembling a device including an ETT and a cuff assembly.

FIGS. 13A-13B illustrates a devices that provide secondary suction at the distal side of the cuff.

FIG. 14 illustrates embodiments of the connecting annular membrane

FIG. 15 illustrates a mold for integral forming of the annular membrane and the sleeve coating.

FIG. 16a and FIG. 16b illustrate an ETT tube including one or more elastic sections.

FIG. 17a and FIG. 17b illustrate a preferred embodiment of a loading mechanism with a wire loop for closing the sleeve proximal end.

FIG. 18a and FIG. 18b illustrate alternative shapes of the sleeve net or weave.

Some embodiments of the present invention relate to methods and apparatus for breathing intubation that were disclosed in PCT/US2009/062227 filed on Oct. 27, 2009 incorporated herein by reference in its entirety. In some embodiments, any combination of features described in the present document and/or in application PCT/US2009/062227 filed on Oct. 27, 2009 incorporated herein by reference in its entirety may be provided.

### DETAILED DESCRIPTION OF EMBODIMENTS

The claims below will be better understood by referring to the present detailed description of example embodiments with reference to the figures. The description, embodiments and figures are not to be taken as limiting the scope of the claims. It should be understood that not every feature of the presently disclosed methods and apparatuses is necessary in every implementation. It should also be understood that throughout this disclosure, where a process or method is shown or described, the steps of the method may be performed in any order or simultaneously, unless it is clear from the context that one step depends on another being performed first. As used throughout this application, the word “may” is used in a permissive sense (i.e., meaning “having the potential to”), rather than the mandatory sense (i.e. meaning “must”).

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

### BRIEF OVERVIEW

Embodiments of the present invention relate to an apparatus, method and kit for reducing a likelihood of fluids leaking from the trachea of a patient undergoing intubation into a lung thereof.

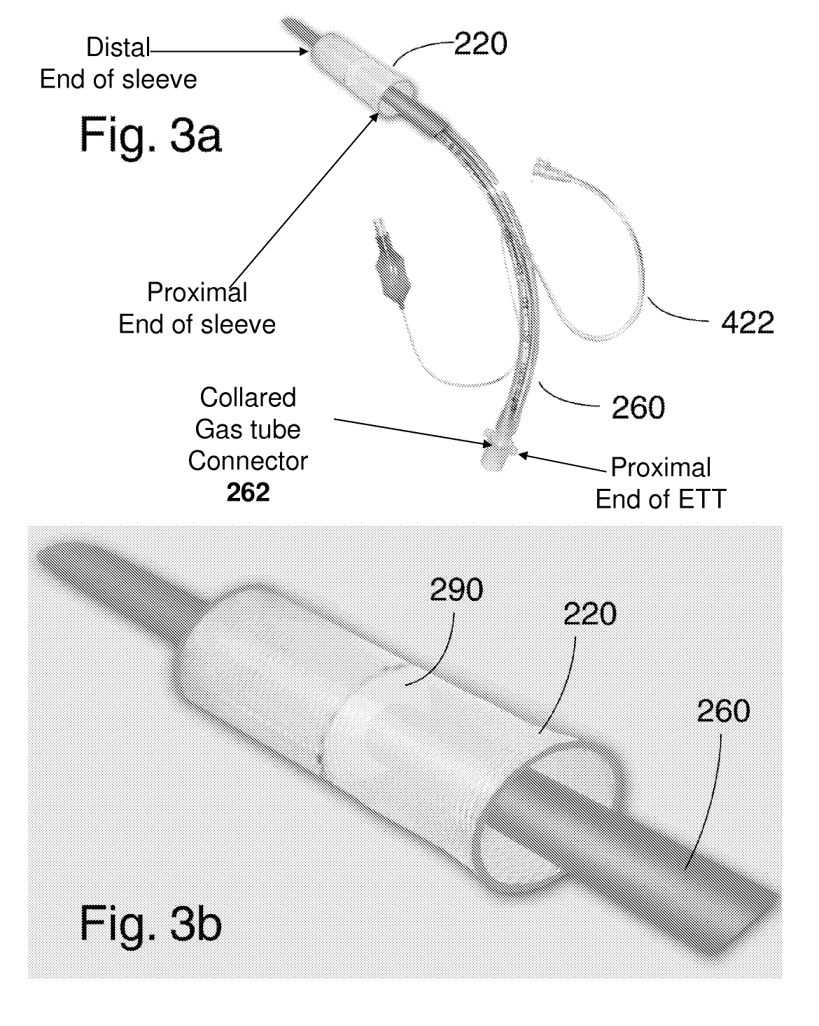

Some embodiments provide a cuff assembly and a related method for preventing and/or hindering a downward motion of fluids in the trachea during intubation. One example of an intubation system comprising a cuff assembly and an ETT is illustrated in FIGS. 3A-3B.

The cuff assembly includes an elastic, outwardly biased, liquid-impermeable elongated sleeve 220 (preferably constructed from a fibrous skeleton coated with a biocompatible elastic material) which, when deployed in the trachea, outwardly presses against the wall of the trachea. The cuff assembly further includes a liquid-impermeable connecting element that is permanently attached to an inner surface of the sleeve.

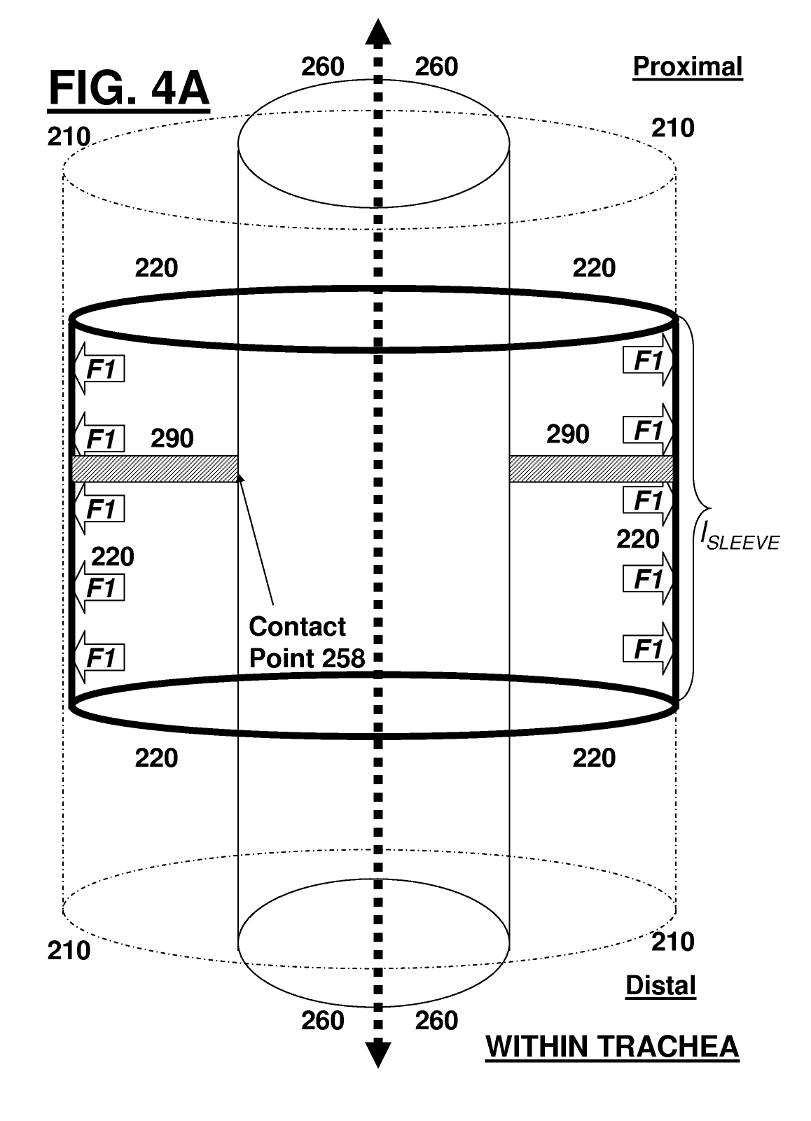

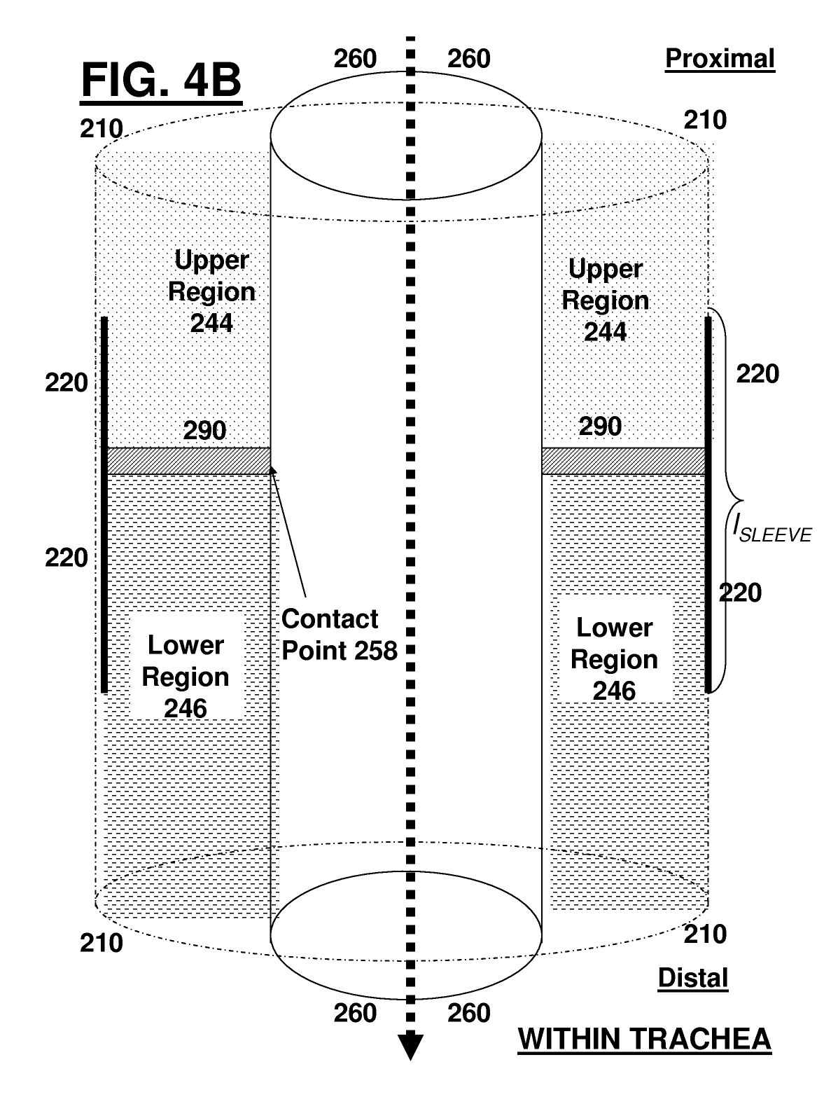

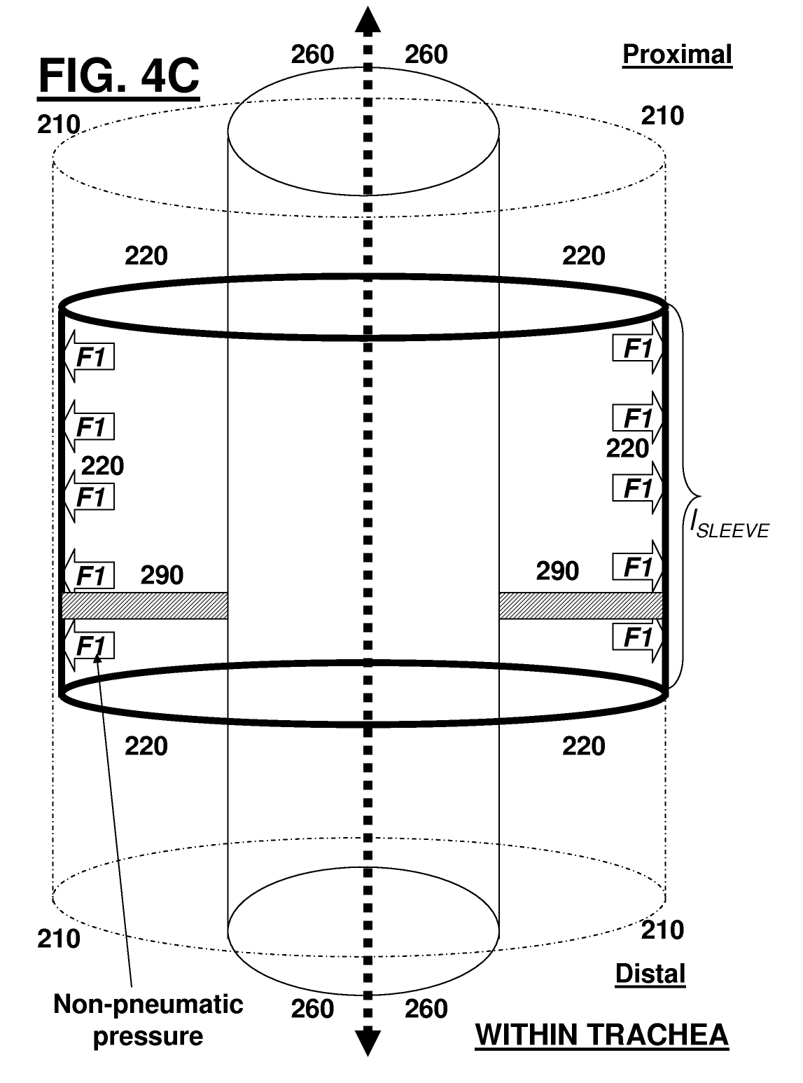

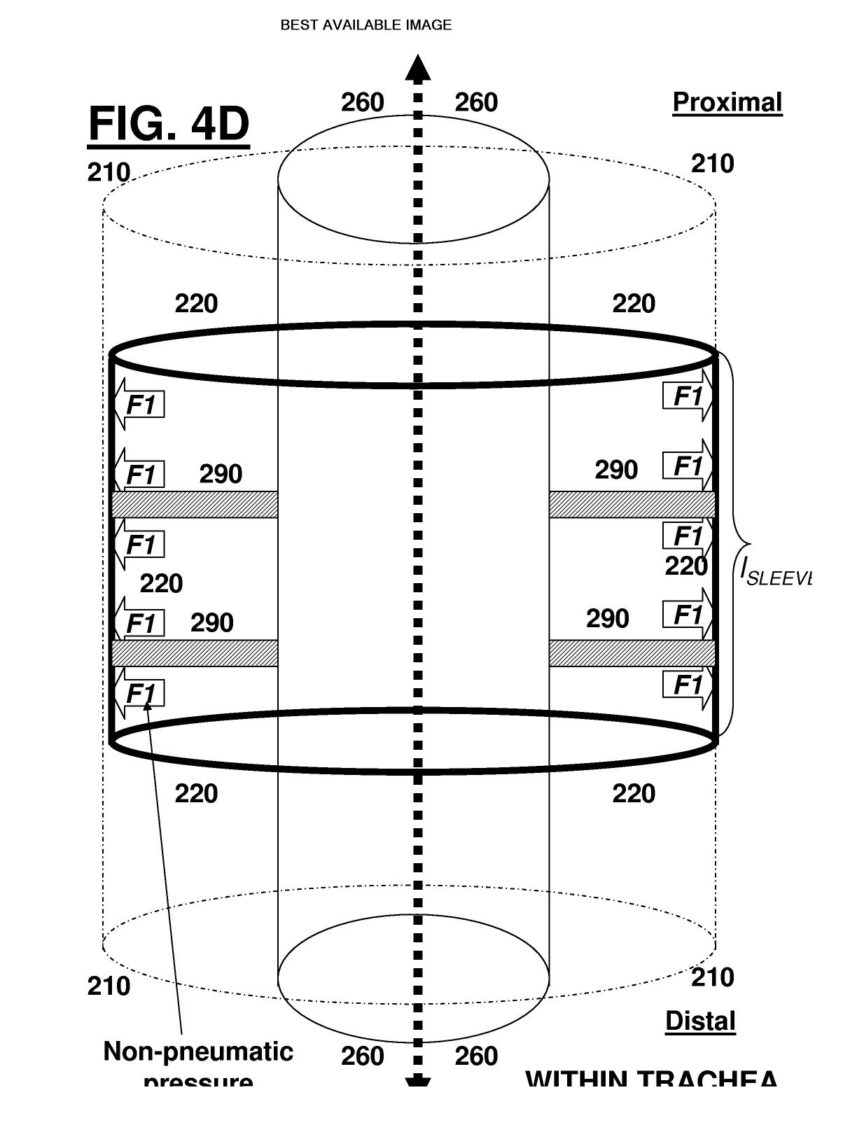

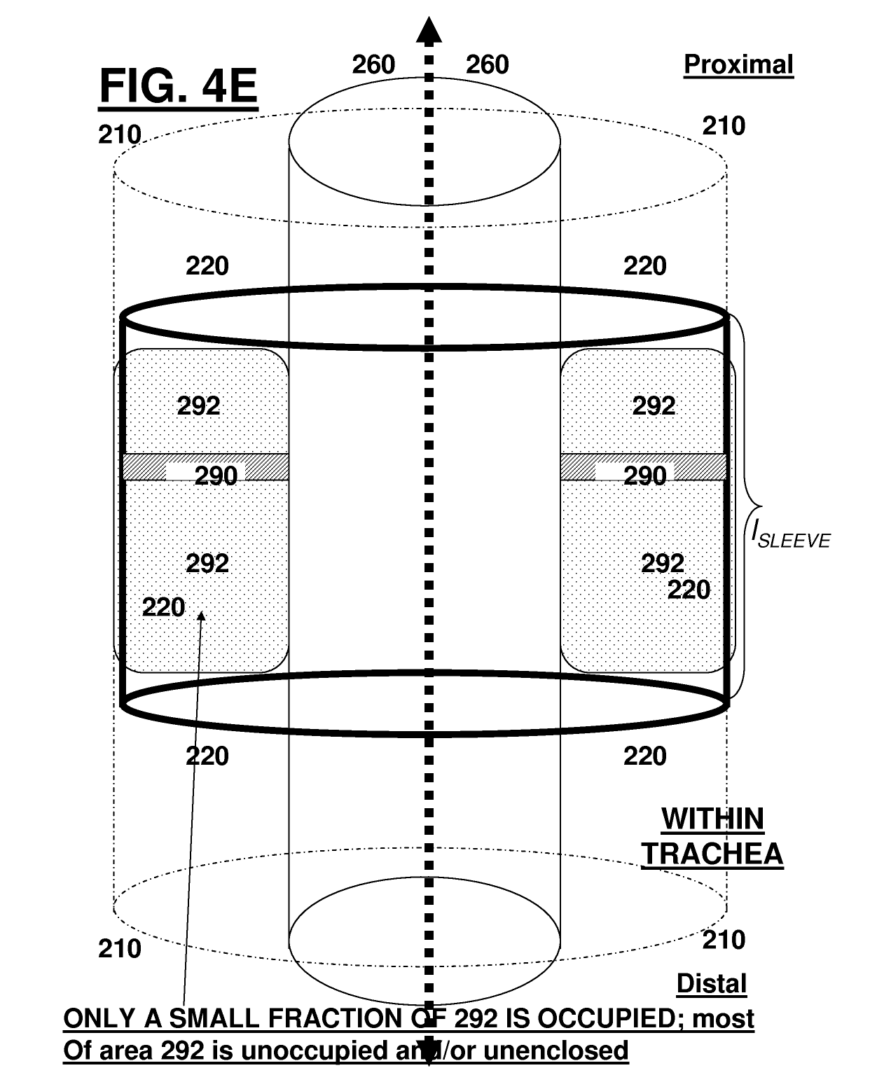

When an ETT 260 passes through an inner region of the elongated sleeve to longitudinally traverse the sleeve 210 (see, for example, FIGS. 3A-3B), the connecting element 290 (for example, comprising one or more liquid-impermeable non-rigid membranes) is in contact with both an outer surface of the ETT 260 and an inner surface of the sleeve 220 (see FIGS. 4A-4E which illustrate the cuff assembly and ETT within trachea 210). This contact may provide a seal, in the interstitial area between the wall of the trachea and the outer surface of the tube, between a proximal portion of the human trachea above the cuff and a distal portion of the human trachea below the cuff.

In contrast to conventional ‘inflation-based’ cuff systems which generally rely on inflation to provide outward pressure on the trachea, embodiments of the present invention may employ an outwardly-biased sleeve constructed from a fibrous skeleton which is coated with a biocompatible elastic material (e.g. silicone, polyurethane or latex). Furthermore, the presence of the connecting element (e.g. an array of membranes) which radially spans the region within the inner surface of the sleeve and outside of the ETT to connect the sleeve to the ETT may be useful for ‘sealing’ an upper region above the connecting element from a lower region below the connecting element, to protect the lungs from downward motion of liquids into the lungs.

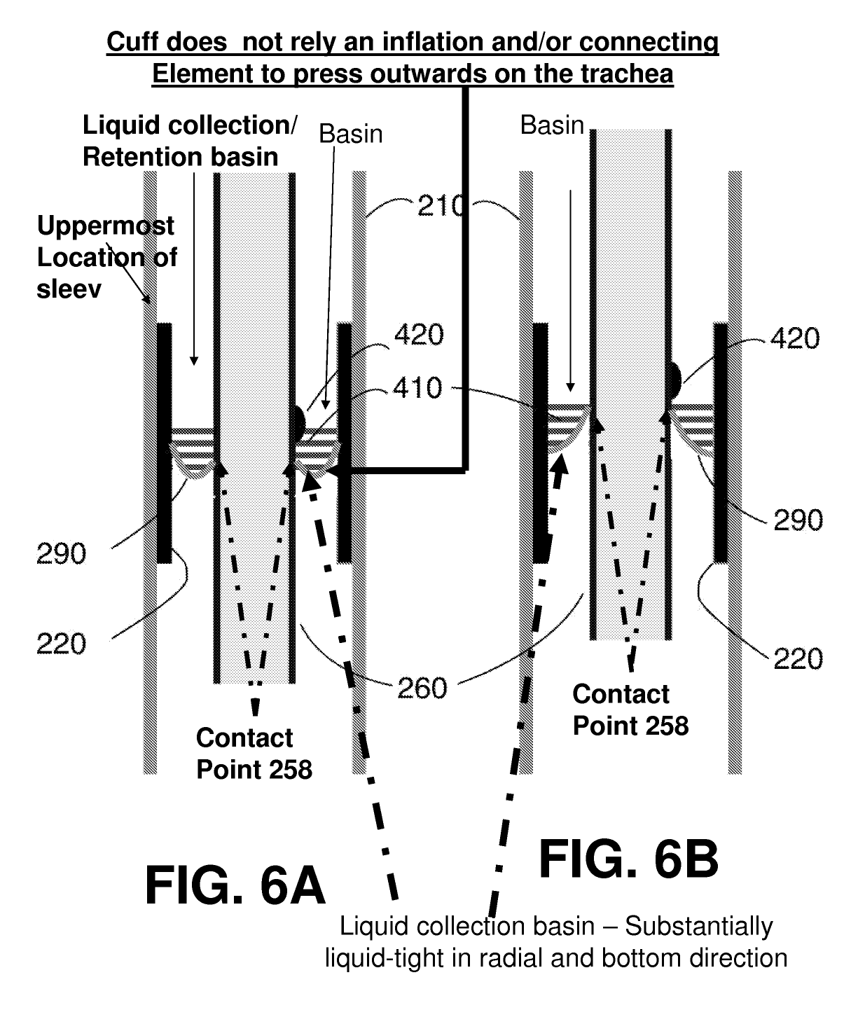

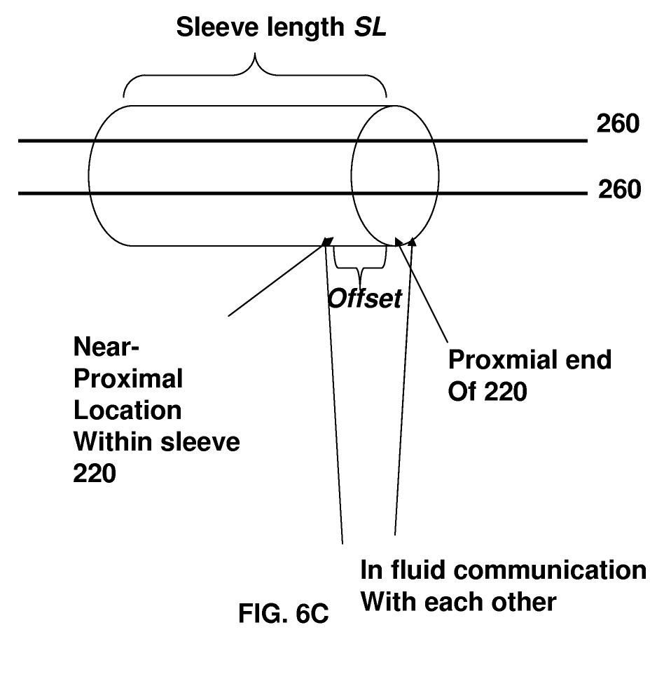

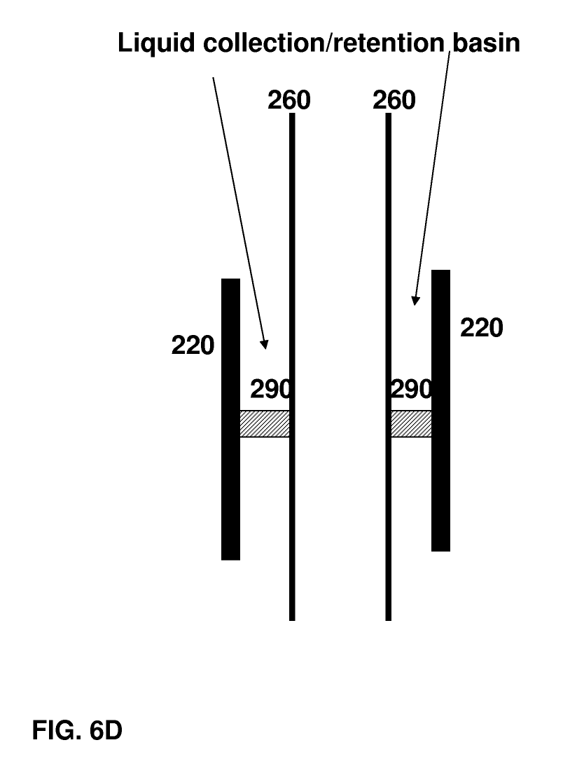

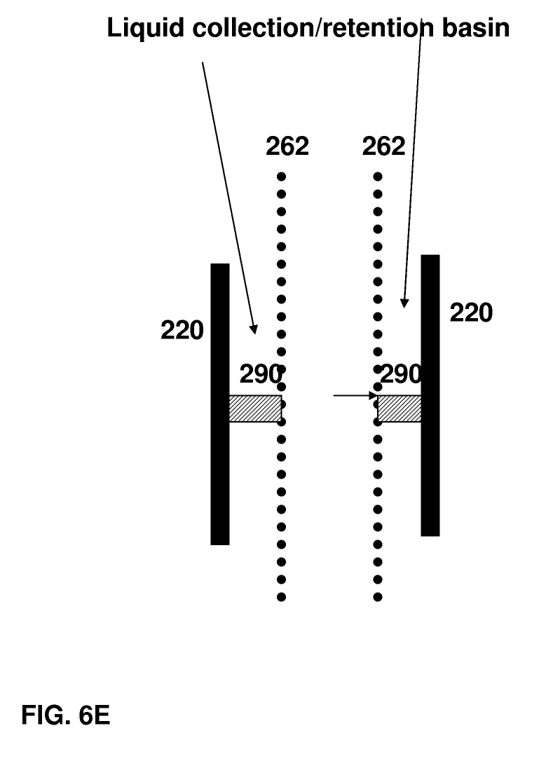

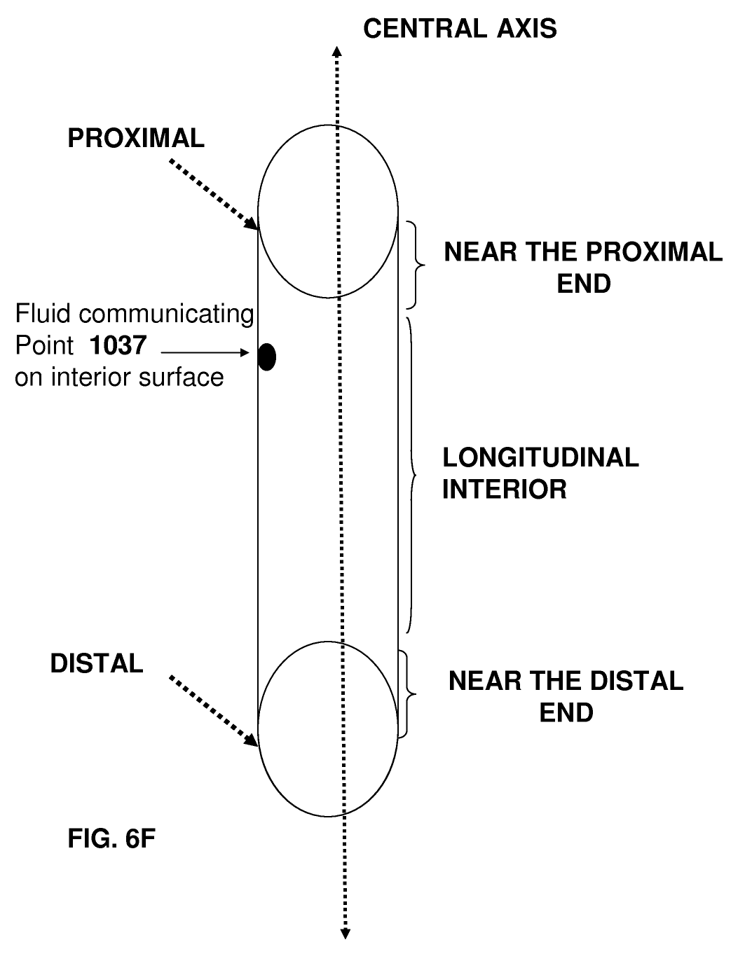

As illustrated in FIG. 6, when the radially-spanning liquid impermeable connecting element 290 spans the region between the outer surface of ETT 260 and the inner surface of sleeve 220, the liquid may collect in a ‘liquid collection’ or retention basin above connecting element 290, rather than penetrating into the lungs. As illustrated in FIGS. 6C-6F, the cuff assembly may be configured so that the point where the connecting element is connected to an interior surface of sleeve 220 is within a longitudinal interior. As will be discussed below, this geometry is useful for providing a ‘large enough collection basin’ for fluids to be collected.

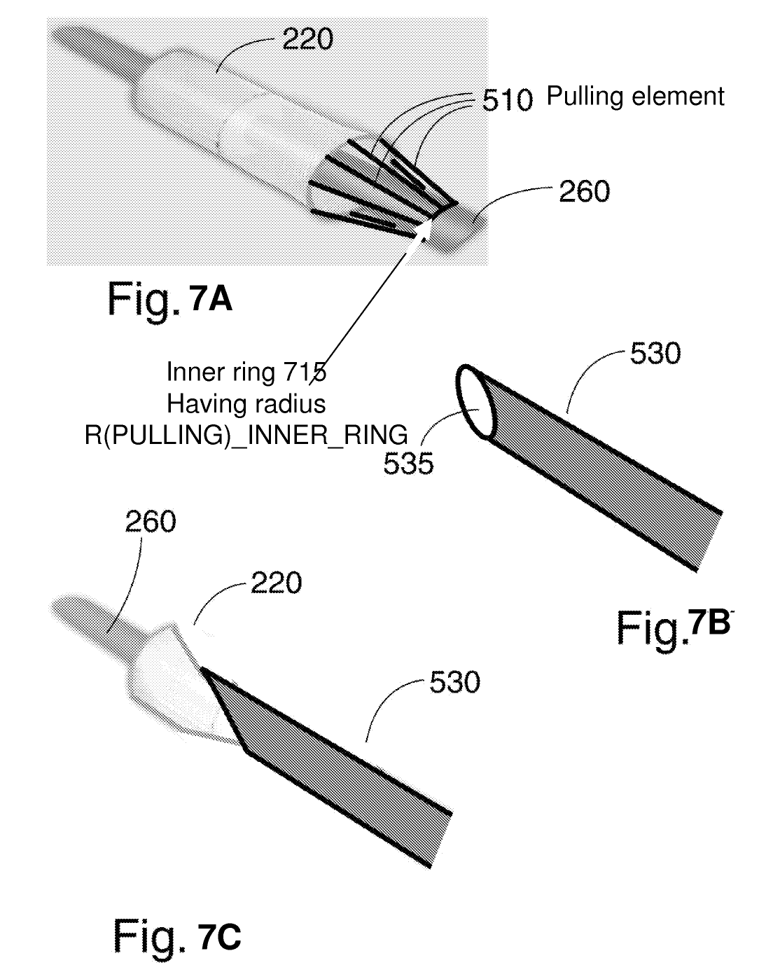

Some embodiments of the present invention relate to kits and/or apparatus for loading the cuff assembly and ETT into the patient's trachea, and to methods of accomplishing the same (see FIGS. 7-10 and the accompanying discussion).

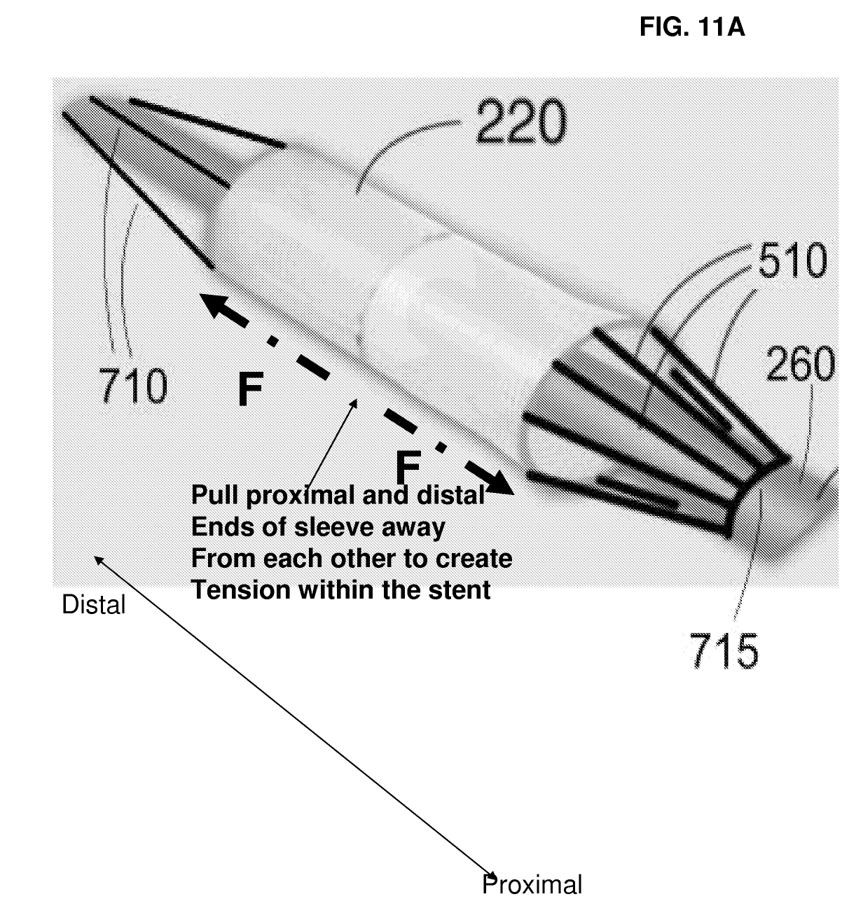

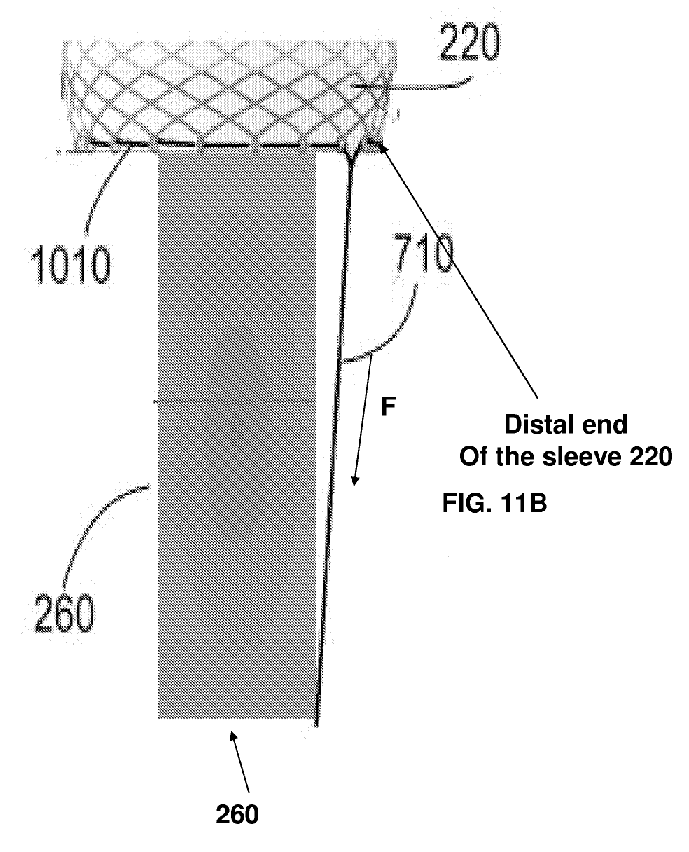

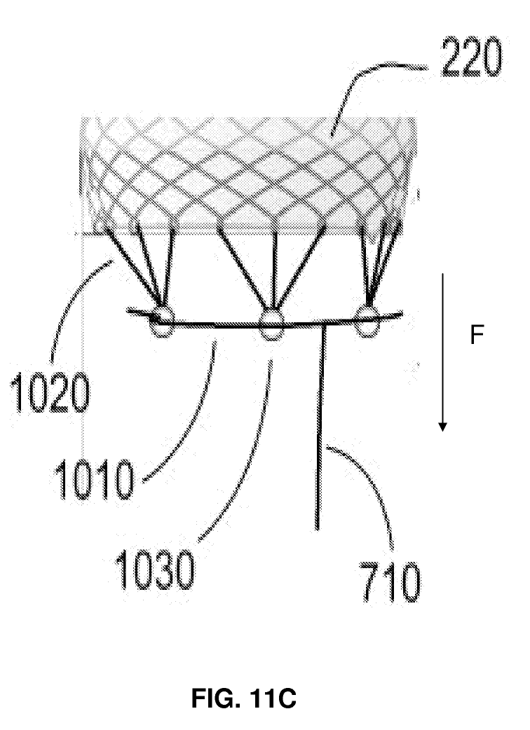

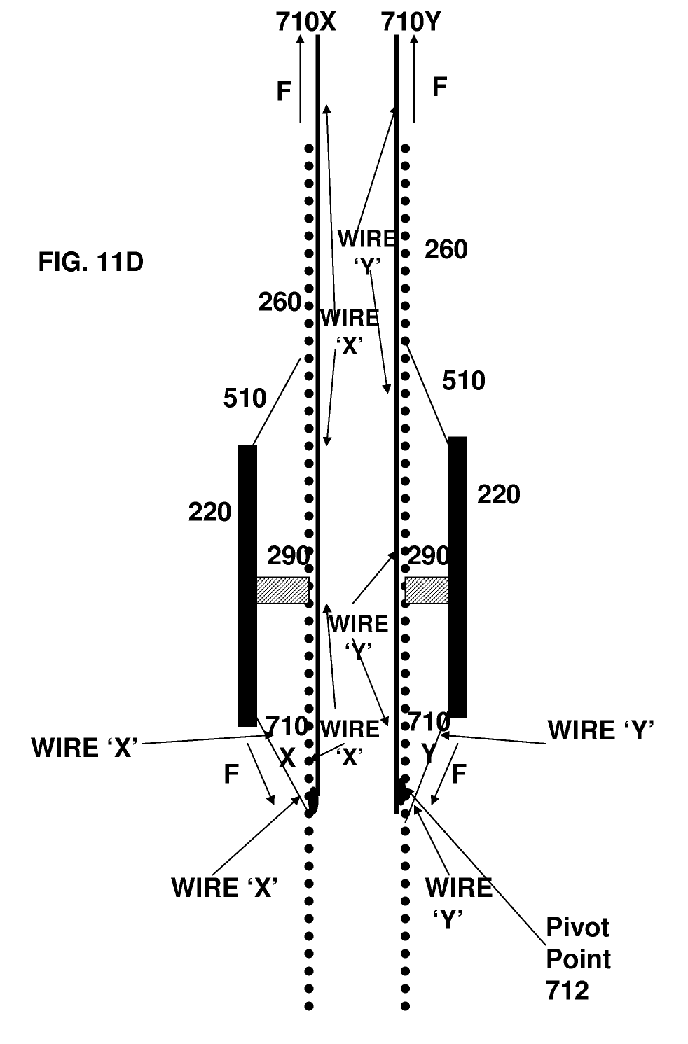

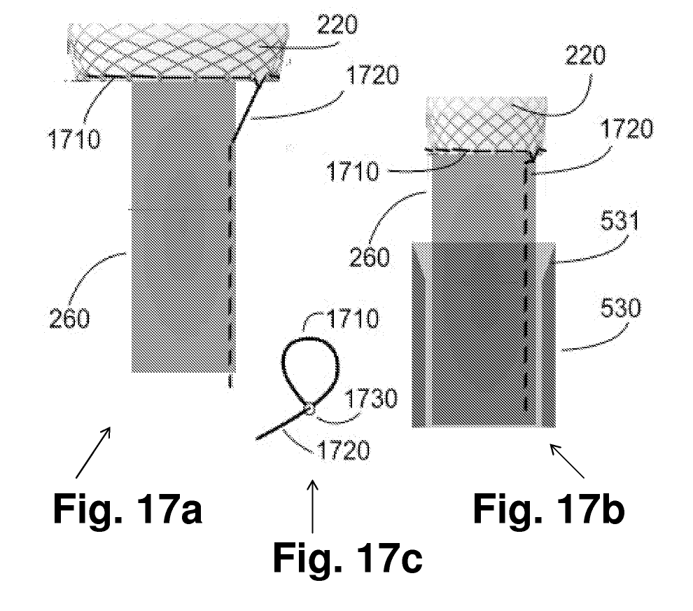

Some embodiments of the present invention relate to kits and/or apparatus for removing the cuff assembly and ETT from the patient's trachea, and to methods of accomplishing the same (see FIGS. 11 and 17 and the accompanying discussion).

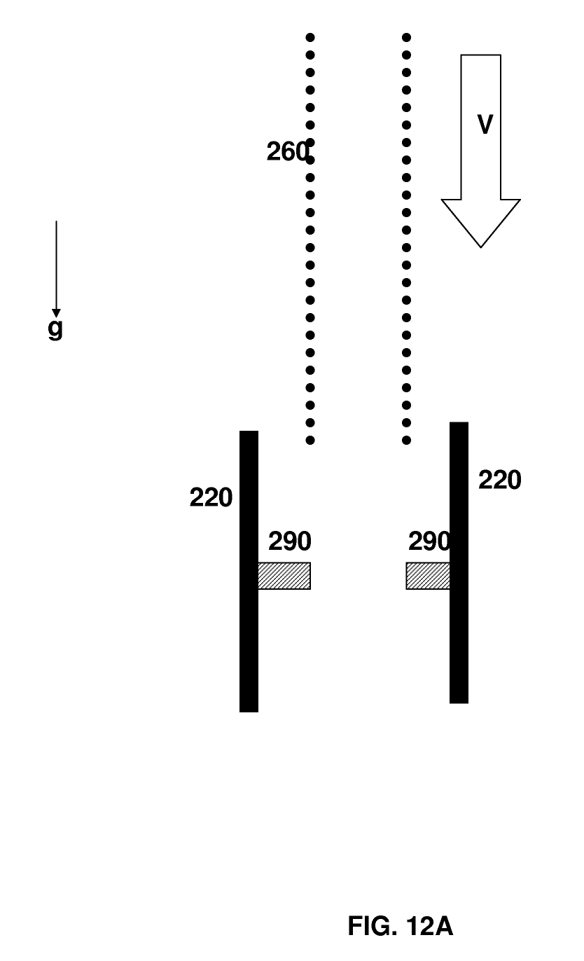

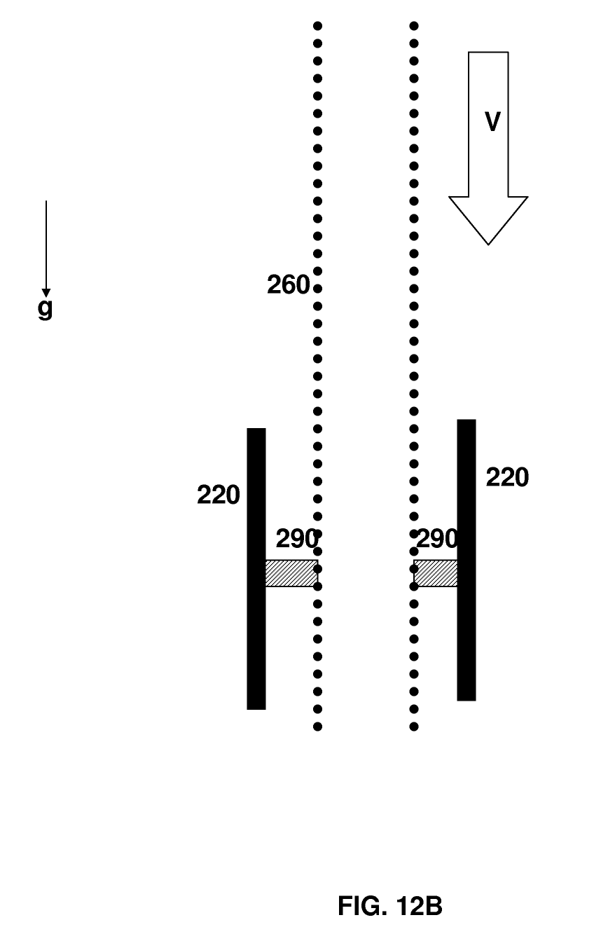

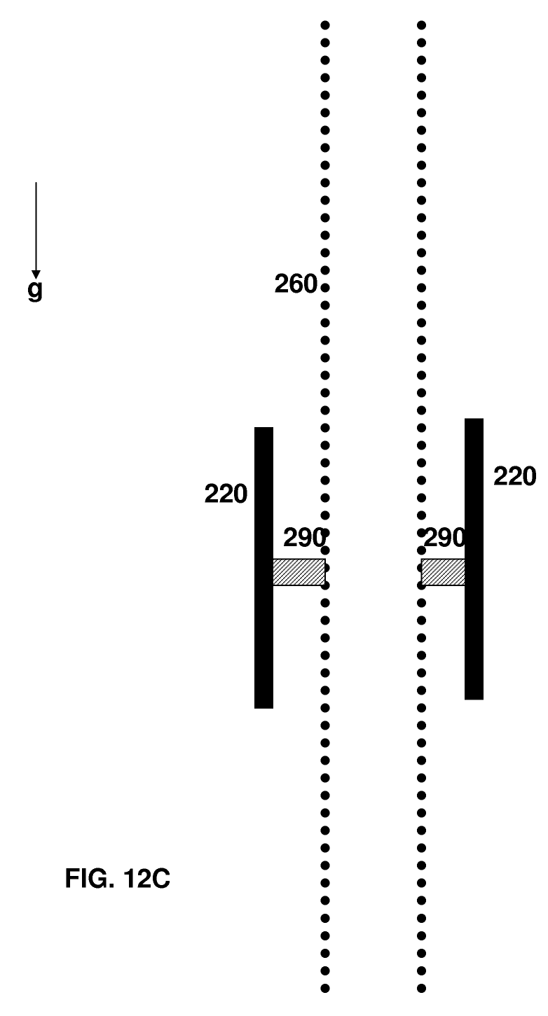

Some embodiments of the present invention relate to kits including the ETT 260 and the cuff assembly, and to methods of assembling the same. FIG. 12 relates to a technique for assembling a kit including an ETT and a cuff assembly so that inward pressure of the connecting element on an outer surface of the ETT retains the cuff assembly to the ETT.

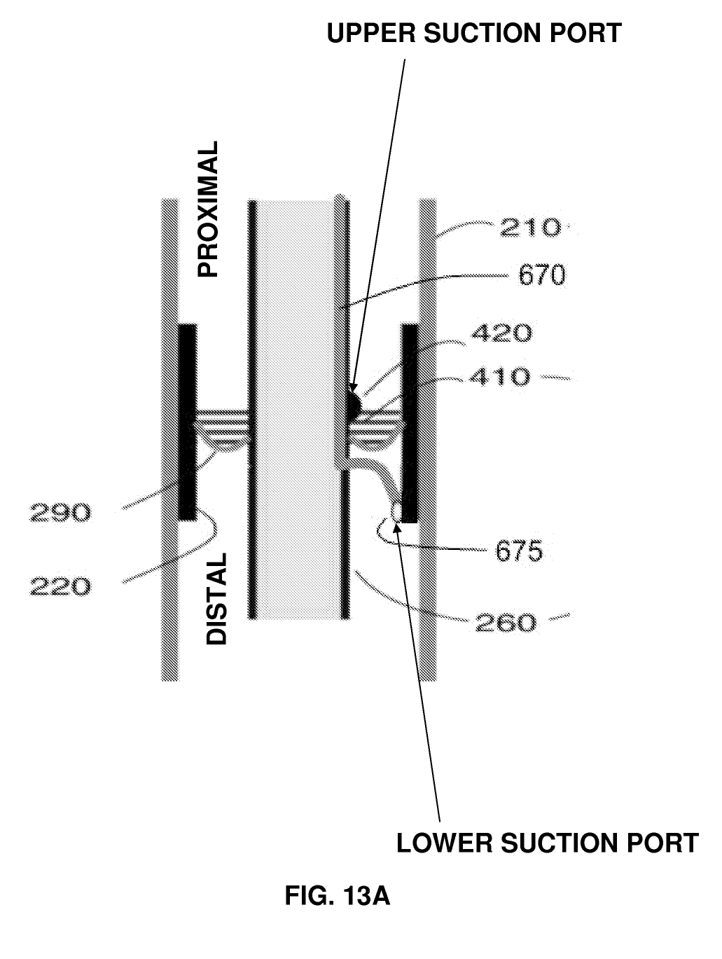

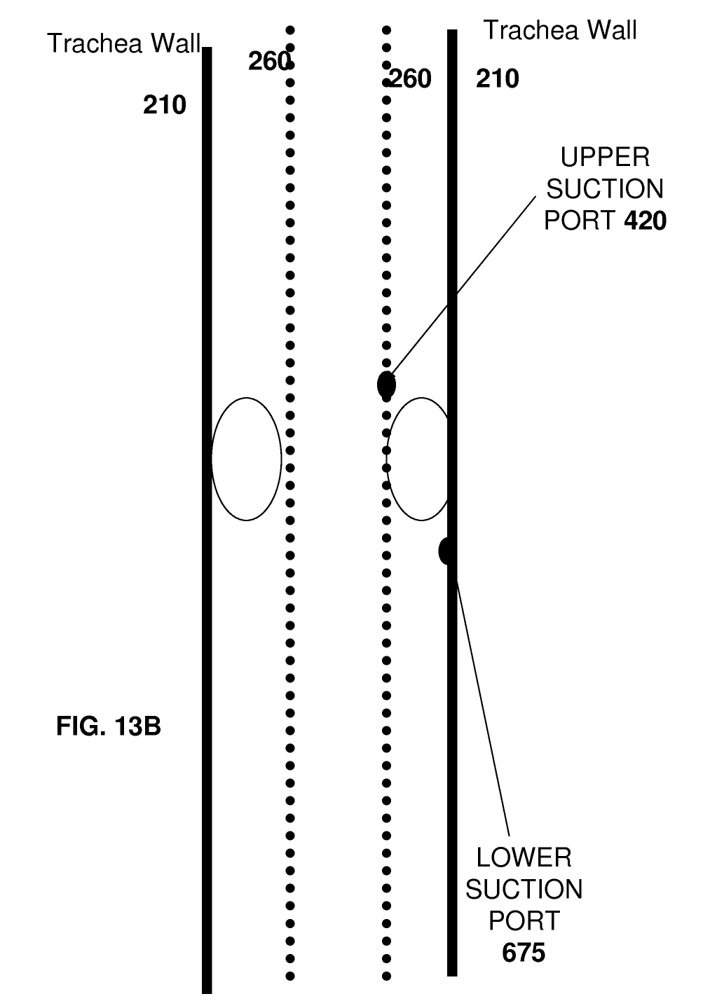

Some embodiments of the present invention relate to techniques and apparatus for providing suction to remove fluids from one or more locations in the trachea (see FIG. 13). In one non-limiting example, the suction device may include a port below the sealing, connecting element.

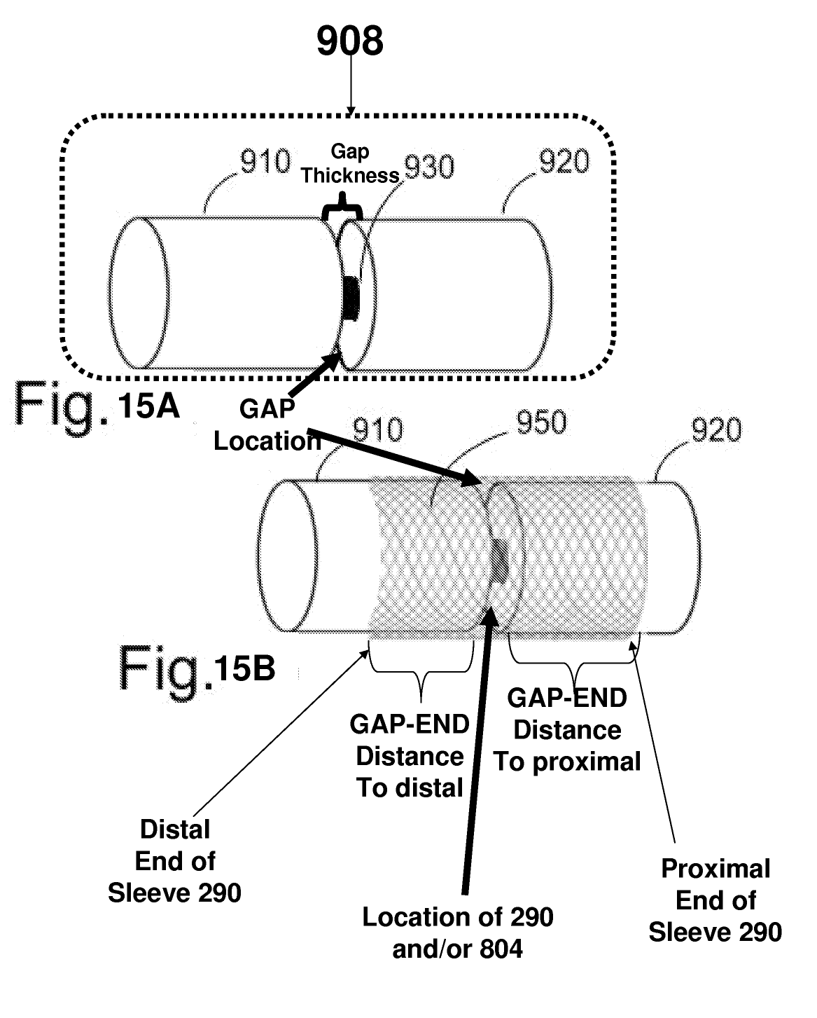

Some embodiments relate to techniques for managing the cuff assembly or a portion thereof (see FIG. 15).

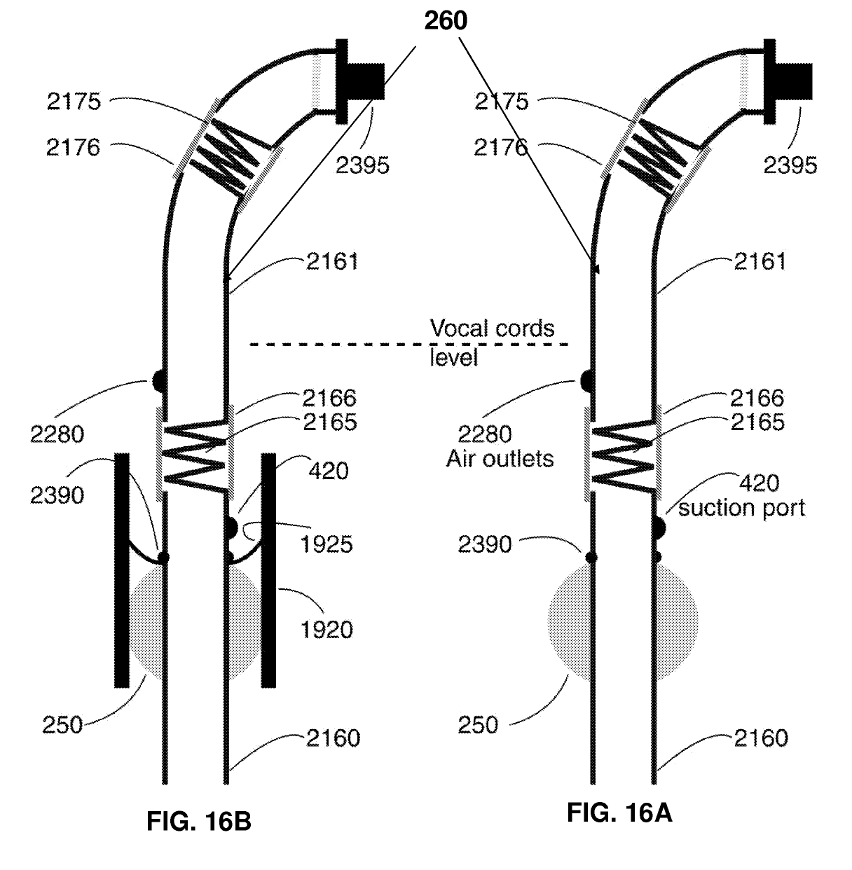

Some embodiments of the present invention relate to an improved ETT, for example, including one or more flexible sections—for example, ‘accordion-like’ sections (see FIG. 16).

Discussion of FIG. 3

FIGS. 3-4, 6-18 illustrate various systems for facilitating ETT and portions of various systems for facilitating ETT. In one example, the system includes an ETT tube and a cuff assembly (or portion thereof) an interior of which is longitudinally traversed by the ETT tube. In some preferred embodiments, the cuff assembly includes an outwardly biased sleeve element comprising a fibrous skeleton coated with and/or covered by a substantially impermeable coating.

Some embodiments relate to kits which, when assembled, provide any ETT system disclosed herein. Thus, for the present disclosure, any disclosed system will also correspond to a respective kit (for example, including any combination of a cuff assembly or a portion thereof, an ETT tube a loading tube, a pulling element, etc). For example, the kit may include packaging which associates the ETT tube with the cuff or a portion thereof—the kit may include instructions for assembling any apparatus or system disclosed in the present application.

In accordance with embodiments of the invention, when the ETT is deployed, it is deployed in such a way that a portion of the ETT contacts a “sleeve” element 220 via a mediating or connecting element, thus creating a seal within the sleeve and between the lungs and the oral cavity. FIGS. 3A and 3B show in cut-away perspective and cross-sectional view along the longitudinal axis, respectively, an example of such a sleeve element 220, constructed and operative in accordance with embodiments of the present invention, deployed in a trachea 210. As shown, sleeve element 220 is of quasi-cylindrical shape, and of length Lₛ. It will also be appreciated that for purposes of the present discussion, sleeve element 220 is an expandable sleeve element in an expanded state; such expandable sleeve elements will be discussed in greater detail below.

As shown in both FIGS. 3A and 3B, the sleeve element 220 may be deployed so that outer surface of sleeve element 220 is in contact with the tracheal tissue; as shown in FIG. 3B illustrates a mediating or connecting element 290 which may radially span the region between an inner surface of the sleeve and an outer surface of ETT 260 in manner that establishes a seal.

In particular, when the pressure exerted by sleeve 220 on trachea 210 (which may itself arise from pressure exerted by balloon cuff 250 on sleeve element 220) is sufficient, in the region 265 between the trachea and the ETT 260 (hereinafter referred to as the “interstitial region”), a seal is created between the lungs on the distal end of sleeve element 220 and the oral cavity on the proximal end of sleeve element 220, thus ensuring that air can be forced into and withdrawn from the lungs through the ETT, as air will be unable to pass into or out of the lungs through the interstitial region. Furthermore, to the extent that fluids enter the trachea, the seal between sleeve element 220 and ETT 260 confines the collected fluids to the region within the sleeve, away from contact with the tracheal wall tissue. For ease of reference, the use of a sleeve together with an ETT may in some places be referred to as “sleeve-supported ETT”.

As noted above, in some embodiments, this outward pressure may be provided by the mechanical properties of sleeve 220, which may be constructed of a fibrous skeleton coated with an elastic coating such that the sleeve is ‘outwardly biased.’ In some embodiments, the outward mechanical pressure provided by the sleeve itself may obviate the need for relying on a balloon-based system and/or relying on ‘inflation of a sealed balloon’ to provide the outward pressure.

The term “sleeve” or “sleeve element” is used so as to intuitively call to mind the image of a generally cylindrical shaped element which can be expanded to press snugly against the inner wall of biological tubes, such as the trachea. A sleeve element may thus be reminiscent of stent devices, but this mental association is not meant to limit the sleeve elements discussed herein to the shapes or designs or constructions of stent devices presently known in the art or to imply that currently known stent devices are necessarily usable in accordance with embodiments of the present invention. Further properties of sleeve elements in accordance with embodiments of the present invention will be discussed further below.

FIG. 3 is an illustration of an intubation device comprising an ETT tube 260 and a cuff assembly deployed to the ETT tube 260. When the intubation device is deployed within the trachea of a patient, the cuff assembly serves to block liquids from penetrating below a location of the cuff assembly within the trachea. The cuff assembly comprises: (i) a substantially-cylindrically shaped sleeve 220 comprising a ‘thin’ wall (for example, including an elastic material so that the sleeve is self-expanding); and (ii) a connecting structure 290 (for example, a flexible connecting structure such as a thin membrane) connecting the ETT tube 260 to the sleeve 220. Both the sleeve 220 and the connecting structure 290 are substantially impermeable to liquids. In one example, the sleeve comprises a fibrous skeleton (for example, a mesh or an array of wires) that is coated by a substantially liquid-impermeable coating or covering layer—for example, according to one or more techniques for constructing a trachea stent discussed above. In one example, the coating 224 or covering layer is an elastic material such as elastic polymer (e.g. polyurethane) or silicone.

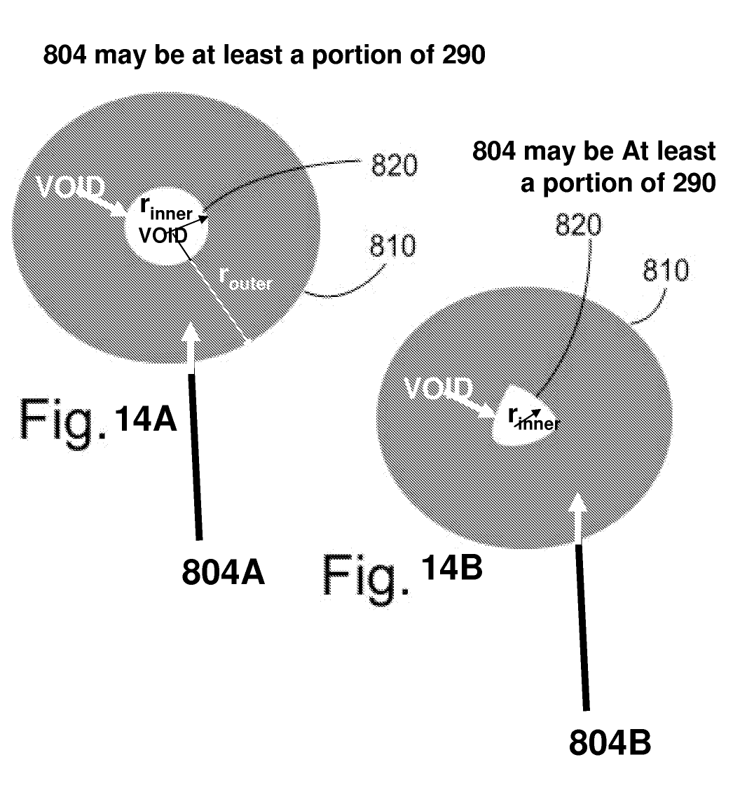

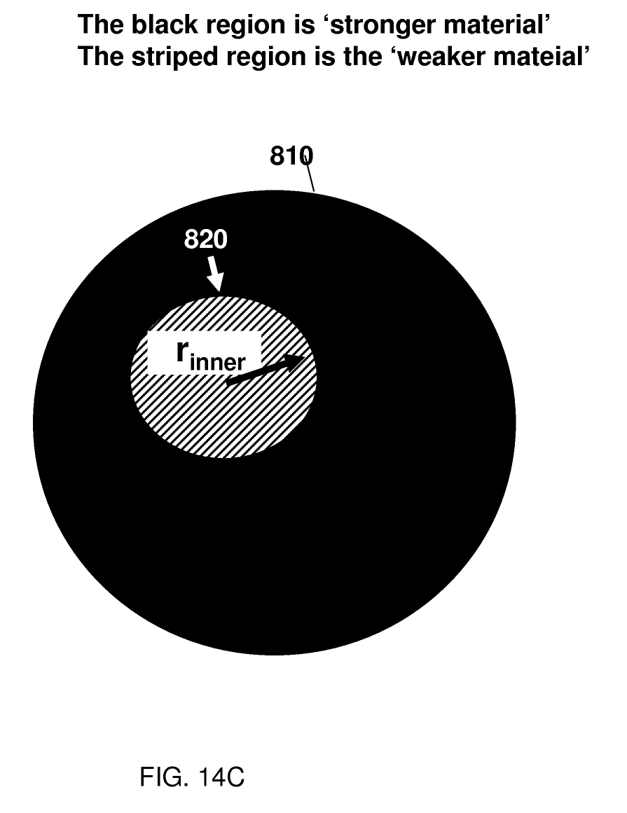

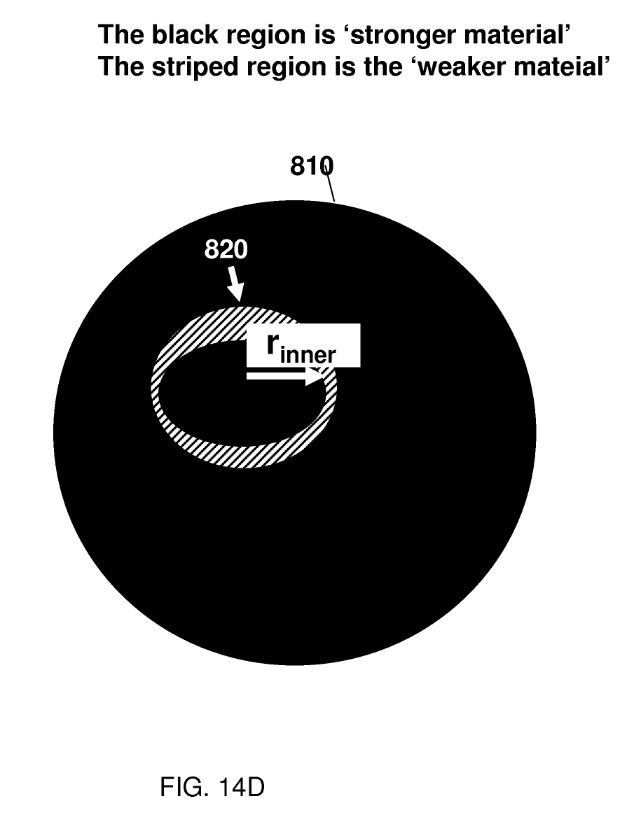

In some embodiments, connecting structure 290 is flexible. As will be discussed below, one example of a connecting structure 290 is an array of one or more thin deformable membranes, preferably also elastic, as illustrated in FIG. 3-4. One non-limiting example of a connecting structure is an annularly shaped structure—for example, as illustrated in FIGS. 14A-14B.

In some preferable embodiments, the connecting structure 290 is ‘permanently attached’ to the sleeve 220. In one example of ‘permanent attachment,’ the connecting structure 290 may be glued or welded to sleeve 220. In yet another example of ‘permanent attachment,’ the connecting structure may be integrally formed with sleeve 220—for example, connecting structure 290 may be molded to sleeve 220, or connecting structure 290 may share a common fibrous skeleton with sleeve 220 or may be integrally formed with coating of sleeve 220.

In some preferred embodiments, the ‘permanent’ link or connection between the connecting structure element 290 and the sleeve 220 is substantially impermeable to liquids.

Furthermore, in some embodiments, the link or connection between the connecting structure element 290 and the ETT tube 260 also needs to be impermeable. Such impermeability can be preferably obtained by tight elastic pressure between an outer surface of ETT tube 260 and an inner surface of the membrane 290 (see, for example, FIG. 12), or by a glue connection.

The combination of the connecting structure 290 (or element) and the sleeve is the ‘cuff’ which hinders and substantially prevents downward motion of liquids within the trachea.

Discussion of FIGS. 4, 6

One salient feature of the example of FIGS. 3-4, is that outward pressure from sleeve 220 is provided without relying on inflation—this is in contrast to a balloon-based cuff where air pressure within the balloon causes an outward force upon the trachea. Thus, it may be said that the cuff of FIGS. 3-4 is ‘non-inflatable’—outward pressure may be provided by the elastic properties of sleeve 220.

In some embodiments, this may obviate the need to provide a ‘balloon inflation lumen’ for inflating the balloon. Typically, prior art devices which rely on a balloon to provide outward pressure upon the trachea to hinder downward flow of fluids include two lumens—a first ‘suction lumen’ for upwardly sucking out fluids that accumulate on the upper or proximal surface of the balloon, and a second ‘inflation lumen.’ In contrast, the device of FIGS. 3-4 do not require any such inflation lumen.

In one non-limiting example, the device of FIGS. 3-4 may include only a single lumen for fluid suction. In another non-limiting example may include multiple fluid suction lumens (see FIG. 13 and the related discussion)—for example, one or more fluid lumens for sucking out fluid on an upper or distal surface of the cuff and one or more fluid lumens for sucking out fluid located below (or distally to) the cuff.

In one non-limiting example, the diameter of the fluid-suction lumen is at least 5% or 10% or 20% and at most 50% or 40% or 30% or 25% a diameter of ETT tube 260.

In an alternate embodiment, the connecting structure element 290 may include an inflatable thin balloon, the length of which is preferably smaller than the length of the sleeve cylinder 220, and/or preferably smaller than half the length of the sleeve cylinder 220.

FIGS. 4 and 6 illustrate an intubation device including a cuff deployed within trachea 210. When the intubation device is deployed within the trachea of a patient, the cuff assembly serves to block liquids from penetrating below a location of the cuff assembly within the trachea.

In the example of FIGS. 4A-4E, the cuff includes (i) an expandable sleeve 220 which, when deployed within trachea 210 exerts an outward force upon trachea 210 (i.e. this may be because the sleeve include an elastic material and an equilibrium/expanded radius of the sleeve 220 exceeds the radius of the trachea 210); and (ii) one or more connecting elements 290. In the example of FIGS. 4A-4E, the connecting element(s) are drawn horizontally—however, this is not a limitation, and as seen in FIGS. 6A-6B, element 290 may ‘sag’ in some embodiments. In accordance with embodiments of the invention (and as illustrated in FIG. 4B), when the ETT is deployed, the combination of the ETT 260, the connecting member 290 and the sleeve 220 substantially creates a substantially fluid-tight seal between (i) an upper region 244 outside of tube 260, within trachea 210 and above connecting member 290; and (ii) a lower region 246 outside of tube 260, within trachea 210 and below connecting member 290.

In some embodiments, the sleeve 210 is self-expanding, and is able to generate a certain amount of outward pressure (i.e. because the sleeve's radius when deployed in the trachea is less than the expanded/equilibrium radius) upon trachea 210. For example, this outward pressure may be in the range of 5-50 cm of H2O and/or at least 5 cm of H2O and/or at most 50 cm of H2O and/or at least 0.5 kPA (kilopascals) and/or at most 5 kPA.

In some embodiments, this outwards force/pressure that is exerted upon a substantially rigid “containing tube” (i.e. having a radius between 0.5 cm and 2 cm—for example, between 0.8 cm and 1.4 cm) in which sleeve 220 is contained is substantially uniform in the theta coordinate (i.e. over the circumference of sleeve 220 and/or containing tube) and/or in the “z” or longitudinal coordinate. In the non-limiting example of FIG. 4, the ‘containing tube’ is the trachea 210.

It is understood that the amount of outward pressure generated by sleeve 220 may be a function of radius of the ‘enclosing tube’ (e.g. the trachea) in which sleeve 220 is deployed may be a function of the size of the enclosing tube—in particular, a function of a difference between the expanded/equilibrium radius R_{EXPANDED} of sleeve 220 and the radius R_{ENCLOSING} of the substantially-rigid ‘enclosing tube’ in which sleeve 220 is located—for example, the radius of trachea 210. For a smaller ‘enclosing tube’ this outward pressure would be greater than for a larger ‘enclosing tube.’

The outward pressure (see F1 in the figs) on the trachea (or any enclosing tube) may depend upon the ‘spring coefficient’—i.e. the ‘material properties’ of that material that the sleeve 220 is constructed from. In some embodiments, the use of the ‘fibrous skeleton’ with a wire or mesh structure (i.e. that is coated with a impermeable elastic material) may be useful for proving the a sleeve that is ‘outwardly biased’ (i.e. when in the trachea, the radius is less than the equilibrium/expanded radius R_{EXPANDED} so that an outward force is exerted).

In one example, for a substantially rigid ‘containing tube’ in which sleeve 220 is contained: (i) the radius of the containing tube is between 0.5 cm and 2 cm—for example, between 0.8 cm and 1.4 cm); (ii) this outward pressure may be in the range of 5-50 cm of H2O and/or at least 5 cm of H2O and/or at most 50 cm of H2O and/or at least 0.5 kPA (kilopascals).

In the example of FIG. 4, the outward pressure is ‘local’—i.e. derived within the ‘thin cylinder’ itself and not as a result of outward pressure of some material (i.e. either solid, liquid or gas) within the substantially-cylindrical shaped volume defined by sleeve 220. Thus, in one example, there is little or no outward force on an ‘inner surface’ of sleeve 220, while there is outward force exerted by the ‘outer surface’ of sleeve 220 on the enclosing tube due to the fact that the sleeve 220 is radially compressed by the enclosing tube (in FIG. 4 this is the trachea 210).

Furthermore, in some embodiments, connector 290 exerts little or no outward force upon sleeve 220.

In the example of FIG. 4A, the connecting element 290 is located near the proximal end (but not at the proximal end) of sleeve 220. In FIG. 4C, connecting element 290 is located more distally—i.e. near the distal end of sleeve 220. In FIG. 4C, connecting element 290 includes a plurality of different membrane elements.

With reference to FIG. 4D, it is noted that, in some embodiments, only a minor fraction of the ‘inter-cylinder’ region (region 292 in FIG. 4E approximates this inter-cylinder region) between ETT 260 and sleeve 220 is occupied by solid material (i.e. occupied by any connecting element 290 and/or by any solid element of the intubation apparatus) and/or only this ‘minor portion’ of the inter-cylinder is out of fluid communication with both a top (i.e. proximal end—for example, where the gas connector is) of ETT tube 260 and bottom (i.e. distal end—for example, where the tapered end and/or Murphey eye of ETT 260) of ETT tube. This ‘minor portion’ may be, for example, at most 40% or at most 30% or at most 20% or at most 10% or at most 5% or at most 3% or at most 2% or at most 1%.

In a first non-limiting example, there is a single connecting membrane 290 (see for example FIG. 4A) whose thickness is about 2 mm. In this example, the length of sleeve 220 is 4 cm, and the ‘minor portion’ or fraction of the ‘inter-cylinder’ region occupied by connecting membrane 290 is 2 mm/40 mm=5%.

In another non-limiting example, it is desired to use a even a thinner connecting membrane (i.e. so that connecting element 290 is even more flexible) and/or connecting element 290 is even thinner—for example, about 0.4 mm. In this example, the ‘minor portion’ is 1%.

FIGS. 6a and 6b show in cut-away perspective and cross-sectional view along the longitudinal axis, respectively, an example of such a sleeve element 220, constructed and operative in accordance with embodiments of the present invention, deployed in a trachea 210. As shown, sleeve element 220 is of quasi-cylindrical shape. It will also be appreciated that for purposes of the present discussion, sleeve element 220 may be an expandable sleeve element; such expandable sleeve elements will be discussed in greater detail below.

As shown in both FIGS. 6a and 6b, the outer surface of sleeve element 220 is in contact with the tracheal tissue; as shown in FIG. 6b, which in addition to sleeve element 220 also shows an endotracheal tube 260 having a connecting membrane 290, a sealing contact is established between a portion of the inner surface of sleeve 220 and outer edge of the membrane 290, which is attached at its inner edge to the ETT 260. Thus, a seal is created between the lungs on the distal end of sleeve element 220 and the oral cavity on the proximal end of sleeve element 220, ensuring that air can be forced into and withdrawn from the lungs. Furthermore, to the extent that fluids enter the trachea, the connecting structure element 290 seal between sleeve element 220 and ETT 260 confines the collected fluids to the region within the sleeve, away from contact with the tracheal wall tissue.

The liquid collection or retention ‘basin’ is defined by the region below the uppermost location of sleeve 220 and above (i.e. on the ‘proxmial side of’ but distal to the uppermost location of sleeve 220) membrane 290.

Thus, in some embodiments, at least one end of sleeve 220 (or both ends of sleeve 220) is substantially free (i.e. for example, at least the ‘proximal end’ of sleeve 220)—i.e. free of membrane 290 and/or configured such that the even when tube 260 longitudinally traverses sleeve 220, the proximal end of sleeve 220 is in fluid communication with a location within sleeve 220 ‘near’ the proximal end (i.e. as in FIG. 6C, a ratio between the distance ‘Offset’ and the length of the sleeve SL is at least 0.05 or 0.1 or 0.2 and a most 0.25 or 0.15 or 0.1). Thus, in some embodiments, there is substantially no fluid obstruction near the proximal end of sleeve 220.

The term “sleeve” or “sleeve element” is used so as to intuitively call to mind the image of a generally cylindrical shaped element which can be expanded to press snugly against the inner wall of biological tubes, such as the trachea. A sleeve element may thus be reminiscent of stent devices, but this mental association is not meant to limit the sleeve elements discussed herein to the shapes or designs or constructions of stent devices presently known in the art. Further properties of sleeve elements in accordance with embodiments of the present invention will be discussed further below.

Reference is now made to FIG. 6D which illustrates the ‘collection basin.’ In some embodiments, it is possible to define the ‘collection basin’ as a portion of the volume contained between ETT 260 and sleeve 220. The ‘collection basin’ is the portion of ‘interstitial volume’ which may retain fluid against the downward force of gravity. The collection basin is (i) below the proximal/top end of sleeve 220; (ii) above (i.e. or proximal to) connecting element 290 which is substantially impermeable to fluids and prevents the fluids from falling ‘deeper’; (iii) within the substantial cylinder region defined by sleeve 220; (iv) outside of ETT 260 and/or outside of a ‘geometrical construct’ 262 2D surface defined by taking the inner circumference/boundary of element 290 (defined by 820 of FIG. 14A-14D) and sweeping this 1D closed curve/boundary 820 longitudinally through space along the central axis of sleeve 220 to define a 2D surface 262 (i.e. analogous to the 2D surface defined by the exterior of ETT 260).

It is similarly possible to define the ‘interstitial volume’ as the region of space that is within sleeve 220 and also either (i) outside of ETT 260; and (ii) ‘outside’ or ‘exterior’ to the region of space defined by the geometrical construct 262 caused by sweeping boundary 820 through space in a direction collinear with a central axis of sleeve 220.

In some embodiments, the fraction of the ‘interstitial volume’ which is occupied by the fluid retention basin (i.e. proximal to the connecting element 290 but below the ‘upper’ or ‘proximal’ end of sleeve—this retention basin may be defined relative4 to ETT outer surface 260 or to the ‘sweeping geometrical construct surface’ 262) is at least 5% or at least 10% or at least 15% of the total ‘interstitial volume’—this ratio may be defined either for the outside of ETT 260 or for the ‘geometrical construct’ surface 262 which is also ‘tube shaped’ but not necessarily cylindrical (see the discussion of FIG. 14B).

Reference is made to FIG. 6F. In FIG. 6F, it is possible to divide the sleeve 220 into three regions: a first region “near the proximal end” of sleeve 220, a second region referred to as the “longitudinal interior” (i.e. distanced from both the proximal and distal end of sleeve 220) and a third region that is ‘near the distal end’ of sleeve 220. In some embodiment, because the connecting element 290 and/or membrane is longitudinally removed from the proximal end, a situation arises whereby a point located both (i) in the longitudinal interior (see FIG. 6F) and (ii) on an inner surface of sleeve 220 is in fluid communication with an outer surface (or a portion thereof) of sleeve 220. This may be because connecting element 290, rather than being located at the proximal end of sleeve 220, is located in the longitudinal interior and/or near the distal end. This “point” in fluid communication with the outer surface of sleeve 220 is referred to in FIG. 6F as the ‘fluid communicating point’ 1037. In some embodiments, a distance between the fluid-communicating point 1037 on the inner surface of the sleeve and each end of the sleeve (i.e. both the proximal and distal end of the sleeve) is at least 10%, or at least 20%, or at least 30% or at least 40% of the sleeve length.

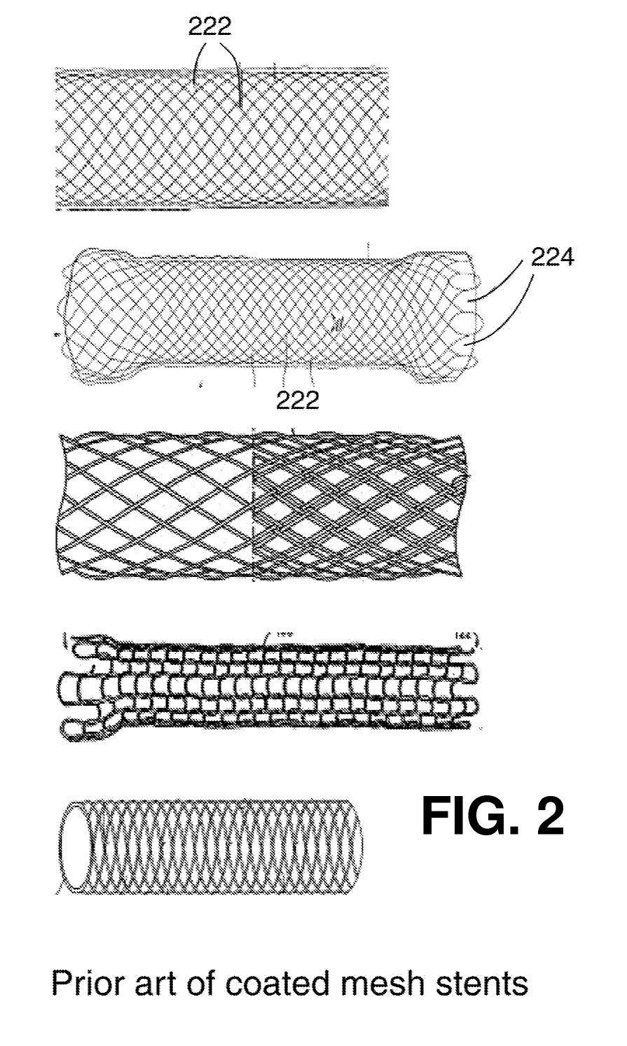

As illustrated in FIG. 2, multitude forms of mesh stents are known in the art, also to be covered with impermeable polymer or silicone layer. The sleeve may or may not have flared ends.

In some embodiments, the sleeve may include some sort of fibrous structure—for example, an array of fibers having a diameter less than 1 mm and/or less than 0.6 mm and/or between 0.1 and 0.4 mm (small). This fibrous structure may be a fibrous skeleton and/or a mesh structure and/or a coil or helix structure and/or a braided structure—for example, as is known for constructing stents. This fibrous structure may be coated with some sort of elastic coating—for example, silicone or a polymer or any other biocompatible elastic coating

In some embodiments, before coating, a certain percentage of the cylinder area is occupied by the fibers—this percentage may be between 1% and 99% of the total area and/or at least 10% and/or at least 20% and/or at least 30% and/or at least 40% and/or at most 70% and/or at most 60% and/or at most 50% and/or most 40% and/or at most 30% of the total area of the ‘cylinder surface’ defined by the fibrous structure which is coated. In one non-limiting example, the ‘occupation fraction’ is between 10% and 30%.

Reference is once again made to FIGS. 6a and 6b. As illustrated in FIG. 6a, use of a sleeve element 220 in accordance with embodiments of the invention enables fluids 410 to pool and collect away from the trachea wall tissue 210; this is in sharp contrast to the known art of ETT tubes in which curvature of the inflated cuff tends to cause fluids 410 to collect exactly at the highest risk location, viz. near the trachea skin tissue 210. As shown in FIG. 6a, in accordance with embodiments of the present invention, the fluids collect at the bottom of a torus-like space, the sides of which are formed by the walls of the sleeve 220 and the ETT 260, and a sealing bottom is formed by the connecting structure element 290. Consequently, even if there are momentary breaks in the seal between the trachea tissue 210 and the sleeve 220, there are no significant volumes of fluids present at the interface between sleeve 220 and trachea 210 which can leak down into the lungs. Such an arrangement may also make the EaTT/cuff/sleeve arrangement less sensitive to the occasional cough or other violent movement of the trachea than the ETT/cuff systems presently in use. Since the fluids are confined away from the trachea wall tissue, momentary breach of the sealed passage to the lungs does not necessarily result in leak of fluids into the lungs.

Moreover, as deployed when used in the prior art, suction elements (which are connected to an external suction tube, not shown, that for example exits the patient's mouth) often cannot reach the contact location between the balloon and the trachea tissue 210 where fluids 410 naturally collect due to the force of gravity, and where suction is most needed. In contrast, as shown in FIGS. 6a and 6b, in embodiments of the present invention, the suction port elements 420 can be placed at locations on the ETT or the sleeve that enable them to collect fluids below the upper end of the sleeve, and thereby preventing fluids from accumulating up to contact the trachea tissue above the sleeve top end. Through the suction port fluids are drawn out via a secondary tube or lumen that eventually leads out to an external draining tube 422 or ‘suction tube’ illustrated in FIG. 3a. For example, a length of the suction tube may be at least 40% or 50% or 60% or 70% of a length of the ETT. In some embodiments, a ratio between a radius of the suction tube and a radius of ETT is at most 0.5, or at most 0.4, or at most 0.3 or at most 0.2. In some embodiments, a distal end of suction tube 422 includes a “gas port” or collar. In the example of FIG. 6B, the ‘bottom of the liquid collection basin of accumulated fluids is near the sleeve—this is not a problem, however, because the connecting element 290 may be attached (typically permanently attached) to the sleeve 220 using a substantially liquid-tight seal.

### Discussion of Various Geometrical Parameters

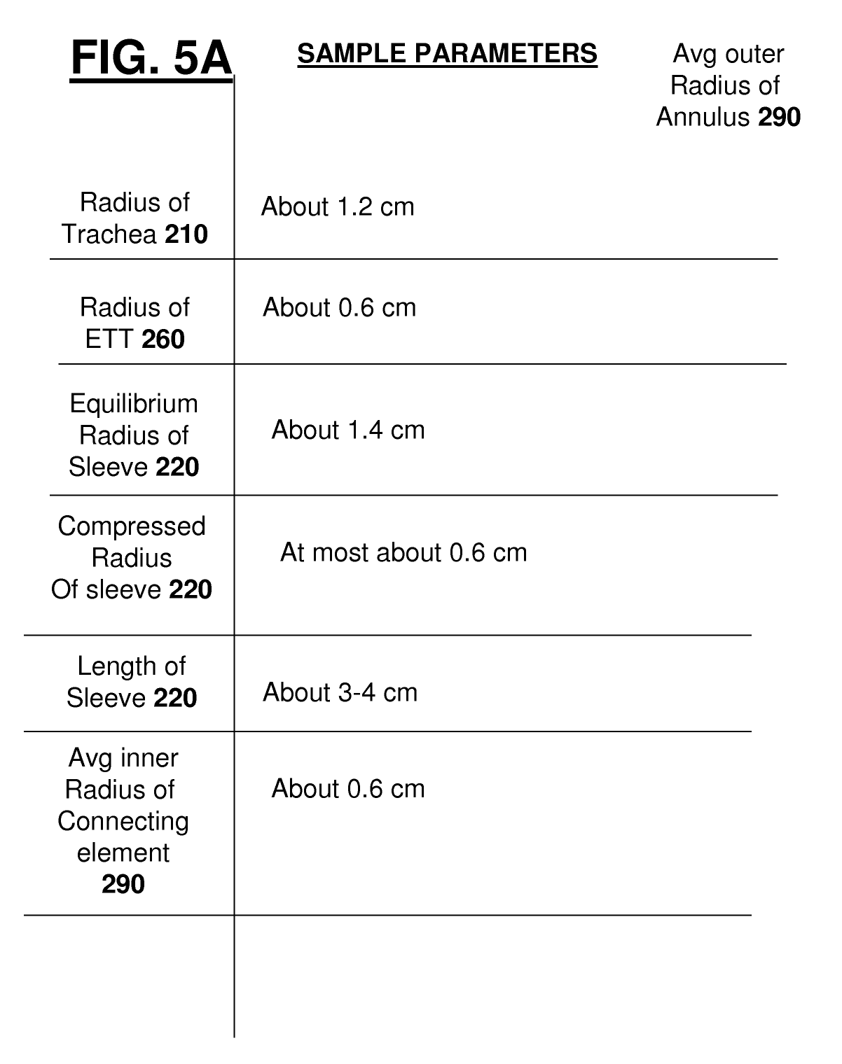

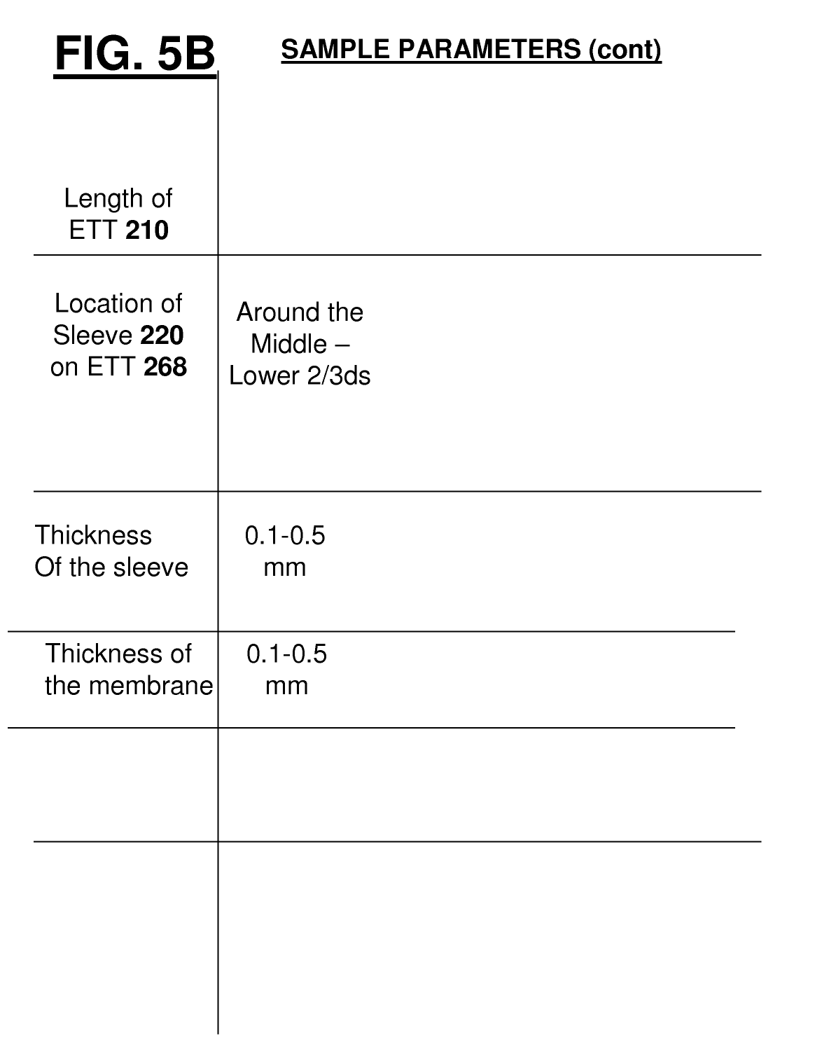

FIG. 5 is a table of some sample parameters associated with an intubation device according to some embodiments. In the example of FIGS. 5A-5B (any combination of parameters or features may be provided including combinations not listed explicitly):

(i) the radius of the trachea is about 1.2 cm (e.g. within a 50% or 30% or 10% tolerance);
(ii) the radius of the ETT 260 is about one-half of the trachea radius and/or about 0.5 or 0.6 cm (e.g. within a 100% or 50% or 30% or 10% tolerance);
(iii) the expanded or equilibrium radius of sleeve 220 is about (i) at least 1.3 or 1.5 or 1.7 times the radius of ETT tube 260 and/or (ii) at most 4 times or 3 times or 2.5 times or 2 times the radius of ETT tube; and/or (iii) between at least 0.8 or at least 0.9 or at least 1 or at least 1.1 or at least 1.2 times the radius of sleeve 220 and at most 2.5 or at most 2 or at most 1.8 or at most 1.6 or at most 1.4 or at most 1.2 times the radius of sleeve 220;
(iv) the expandable/elastic sleeve 220 is capable of compressing to a radius that is at most 1.5 or at most 1.3 or at most 1.1 or at most 1.0 or at most 0.9 or at most 0.8 times a radius of ETT tube 260 and/or to a radius that is most 80% or at most 70% or at most 60% or at most 50% times an equilibrium/expanded radius of sleeve—in some embodiments, it is the elastic coating that provides this property
(v) a length of the sleeve that is between 2 and 6 cm (or between 2 cm and 8 cm) and/or at least 3% or at least 5% or at least 7% or at least 10% or at least 15% or at least 20% of a length of ETT tube 260 and/or at most 40% or at most 30% or at most 20% or art most 15% or at most 10% of a length of ETT tube;
(vi) the connecting element 290 (see, for example, FIG. 14) may in some embodiments have an average inner radius (i.e. the inner surface may not necessarily be circular—for example, it may be triangularly shaped as in FIG. 14B) that is substantially equal (i.e. within a tolerance of 10% or 30% or 50%) to a radius of ETT 260. The average inner radius or AVG(R^{ANNULUS}_{INNER}) may be at most 80% or at most 70% or at most 60% or at most 50% of the ‘outer radius’ R^{ANNULUS}_{OUTER} of connecting element 290 (or of sleeve 220) the average outer radius and/or the sleeve 220 radius. The average inner radius (or AVG(R^{ANNULUS}_{INNER}) may be at least 15% or at least 20% or at least 30% or at least 40% or at least 50% of the ‘outer radius’ R^{ANNULUS}_{OUTER} of connecting element 290 (or of sleeve 220) the average outer radius and/or the sleeve 220 radius. The ‘inner radius’ refers, when the connecting element is substantially annularly shaped to provide at least one of (i) a void within a substantially disk-shaped connecting element or membrane 290, to the radius of the ‘embedded void’ near the center of the ‘disk.’; (ii) a boundary of a region where the membrane is weaker to provide a breaking point (or a ‘pre-determined breaking outline) which may be relatively easily penetrated by a substantially blunt object—for example, an end of a hollow plastic tube of any shape—for example, a distal end of an ETT tube.

As will be discussed below, the entire region between the inner boundary defined by 820 of FIG. 14 and the outer boundary defined by 810 may be substantially impermeable to liquids—thus, the membrane may be at least partially obstructing (i.e. either (i) only partially obstructing where there is a void as in FIGS. 14A-14B or (ii) partially or completely obstructing as in FIGS. 14C-14D) and locally, in the region outside of the inner boundary 820 (and within the outer boundary 810) may be completely obstructing to liquids.

(vii) in some embodiments, the ETT tube may have properties of a ‘standard ETT tube,’—i.e. having a substantially constant circumference, at least partially bendable—the ETT tube does not have to be round, as is discussed below with reference to FIG. 14B. Convention or standard ETT 1260 tubes typically have a length of between 20 and 50 cm. In different embodiments, the length of the ETT tube may be at least 20 cm and/or at most 100 cm or at most 80 cm or at most 60 cm or at most 50 cm.
(viii) the location of the ‘longitudinal mid-point’ of sleeve 220 is in the lower ⅔ of ETT tube 260—for example, between 40% and 80% or between 50% and 70% of the distance between (a) the top/proximal end of ETT tube and (b) the bottom/distal end of ETT tube 260;
(ix) sleeve 220 and/or any membrane of connecting structure 290 may be ‘thin’—e.g. at most 1 mm and at most 0.8 mm and/or at most 0.6 mm and/or at least 0.1 mm and/or at least 0.2 mm and/or at least 0.3 mm;
(x) sleeve 220 is part of a ‘substantially balloonless’ system—i.e. there is no airtight or liquid-tight compartment within sleeve 220 which occupies more than 10% or more than 30% or more than 50% or more than 70% or more than 80% or more than 90% or more than 95% of a volume of the substantially cylindrically shaped region of space defined as the interior of sleeve 220—in some embodiments, connecting element 290 may also not be part an exterior of an airtight or fluid tight compartment.
Techniques for Loading the Cuff onto the ETT Tube and/or for Deploying the Cuff into a Patient's Trachea and/or Removing the Cuff from the Patient's Trachea

In accordance with some embodiments, the sleeve may be incorporated for delivery on top of the ETT itself. Variations of such embodiments may include direct attachment of the connecting structure membrane element 290 to the ETT. Such an arrangement renders the sealing independent of pressure.

FIG. 7-11 relate to a technique for closing the sleeve around the ETT tube before deploying the sleeve/cuff (including the sleeve) combination into a patients trachea, or for removal of the sleeve/cuff combination out of a patients trachea. As shown in FIG. 7a, two or more wire like elements 510 extend at one end from the top end of the sleeve 220 and are attached at their other end to the tube 260. In the example of FIG. 7A an array of parallel wires are illustrated (i.e. as part of the ‘pulling element discussed below)—however, this is not a limitation, and pulling element 510 may have other physical properties—for example, in some embodiments, pulling element may include a net or mesh which is permeable to fluids. In one example, these wires can be continuations of the wires 510 composing the braid of the sleeve 220. In other preferred embodiments, the wire like elements 510 may be extensions of the coating layer (e.g., silicone) of the sleeve—for example, an array of strips of the material which coats the fibrous structure of sleeve and extends beyond the sleeve. In yet other embodiments the wire elements 510 may be glued or stitched to the sleeve 220.

FIG. 7B illustrates a loading tube part 530. The tube 530 has an inner lumen 535 with a diameter which is just slightly bigger than the diameter of when the sleeve 220 is tightly wrapped around the tube 260 (i.e., close to the thickness of the sleeve 220 added to the outer diameter of the breathing tube 260).

It is appreciated that when it is written that the radius of loading tube 530 R^{LOADING}_{TUBE} is “substantially equal” to the radius of the ETT tube R^{ETT}_{TUBE} this refers to the inner radius of loading tube 530 R^{LOADING}_{TUBE} being substantially equal to the outer radius of the ETT tube R^{ETT}_{TUBE} as illustrated in FIG. 7 (i.e. within any specified tolerance).

As illustrated in FIG. 7C, when the loading tube 530 is lowered around the breathing tube 260, it comes to press on the wires 510 which in turn pull on and compress down the sleeve 220 (i.e. since wires 510 provide a radial inward force on sleeve 520 to move sleeve 220 radially inwardly to ETT 260). Thereby, the loading tube 530 is encircling and confining the sleeve 220 within its lumen 535 to a tight configuration. Eventually the loading tube 530 is lowered along to cover the full length of the sleeve 220.

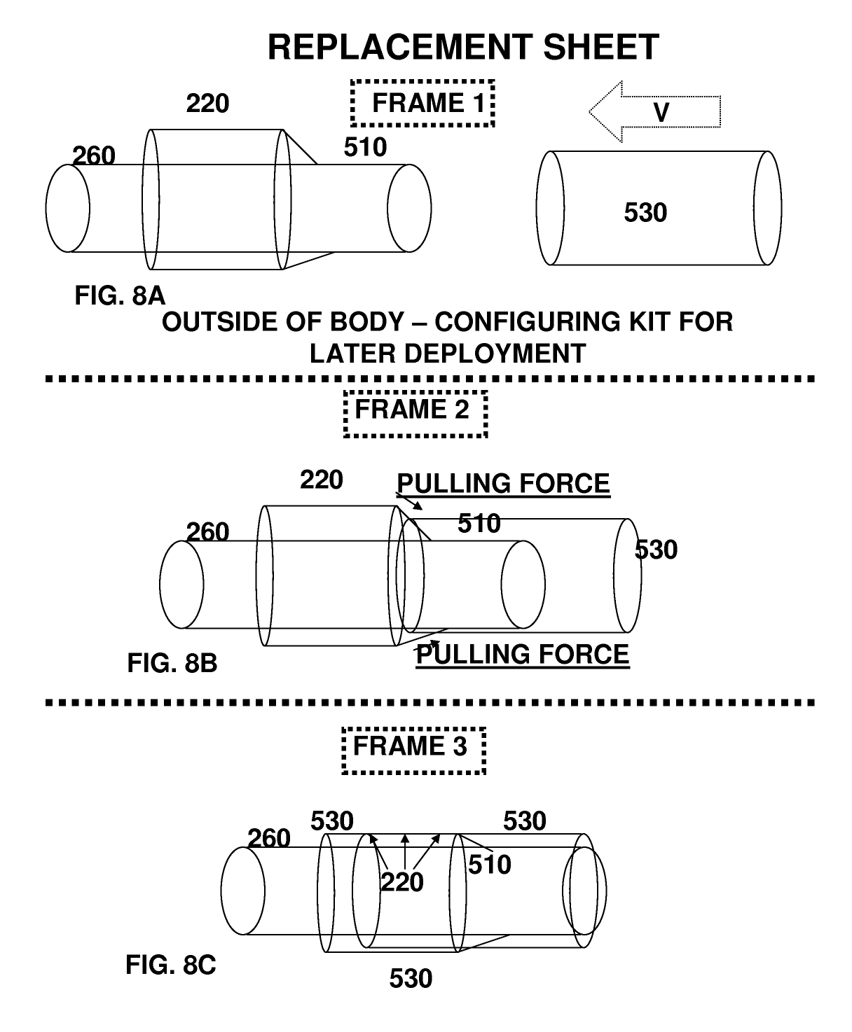

FIG. 8 illustrates a sequence of frame illustrating how it is possible to slide loading tube 530 over sleeve 220 to compress sleeve 220 to ETT tube 260. In frame 1, the loading tube 530 is moved towards ETT tube. In frame 2, when the loading tube reaches the ‘pulling element’ which is at an angle, a longitudinally downward force on pulling element 510 causes pulling element to exert a radially inward force on sleeve 220. IN frame 3, loading tube 530 reaches the distal end of sleeve 220.

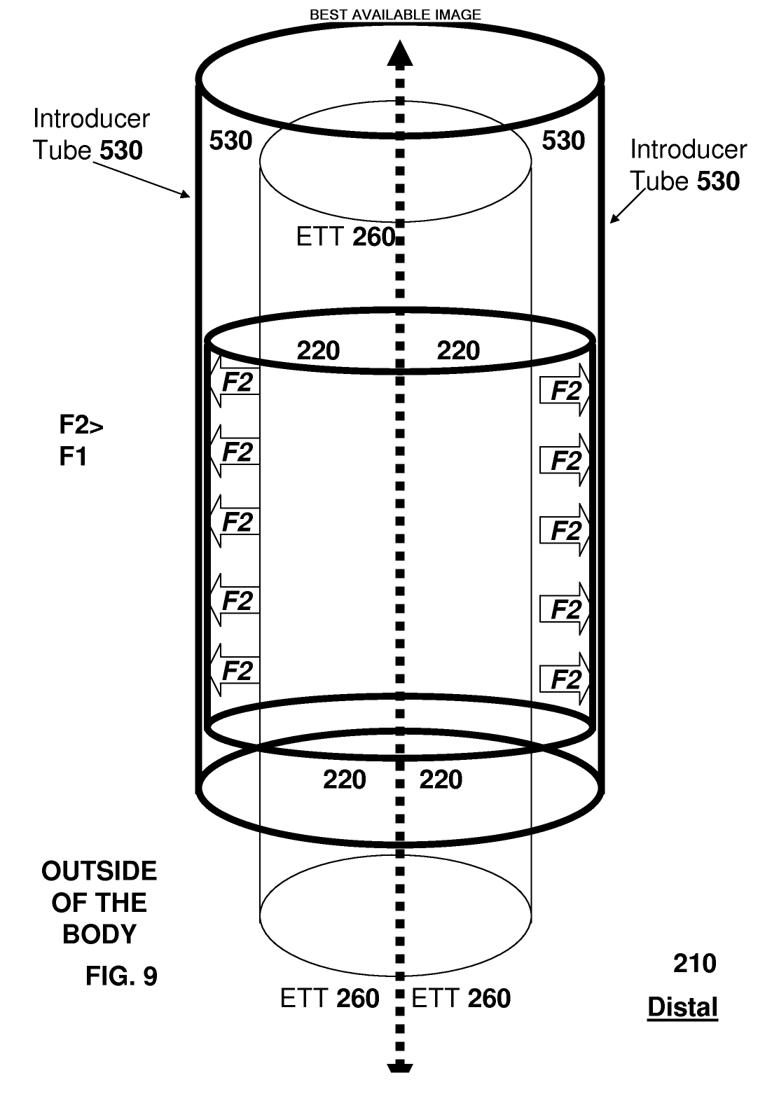

As illustrated in FIG. 9, when the sleeve is within loading (or; ‘introducer’) tube 530, the outward force/pressure upon loading tube exceeds the outward force/pressure on trachea 510 (or an equivalently sized ‘containing tube’ as discussed above) by a factor of at least 1.2 or 1.5 or 1.8 or 2 or 2.5 or 3. Loading tube 520 is only slightly bigger than ETT tube 260.

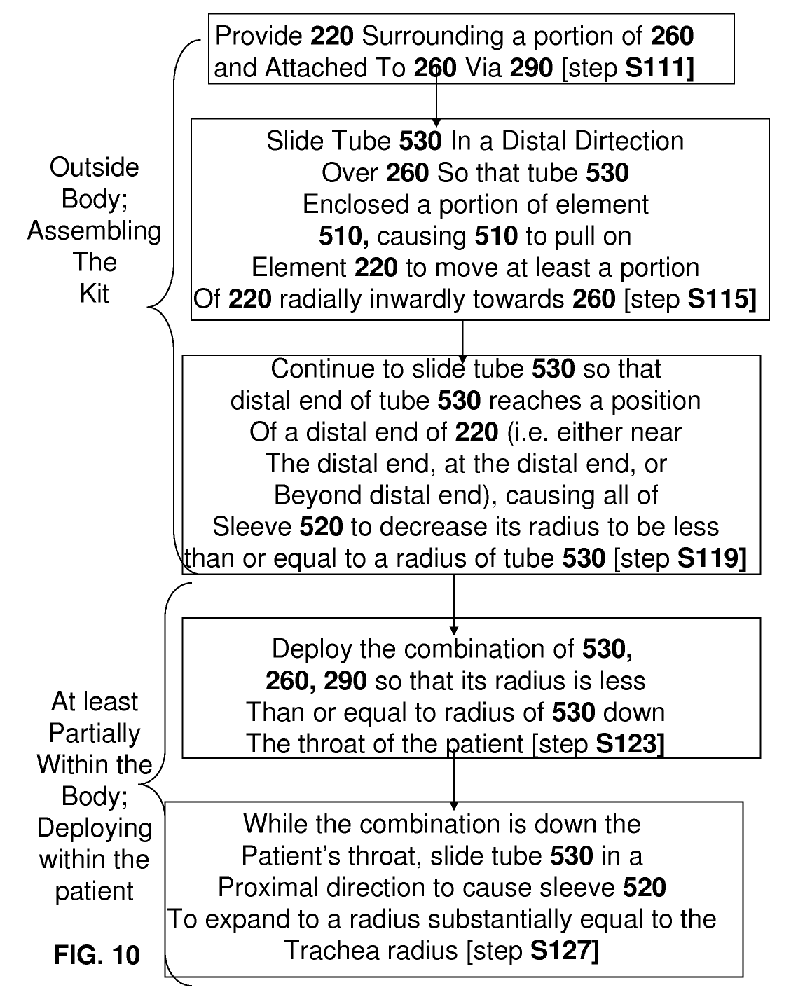

FIG. 10 is a flow chart of a technique for deploying ETT tube 260 and sleeve 220 into the trachea. In steps S111-S119, a ‘kit’ of the loading tube 520 and the cuff/ETT tube combination is assembled to compress sleeve 220 to ETT tube 260. In steps S123-127, the combination of the loading tube, the ETT tube and the cuff are inserted into the patients trachea. In steps 123-127, the loading tube is withdrawn from the trachea while the ETT tube and cuff remain in the trachea—this cause the sleeve 220 to expand outwardly to the trachea to create a substantial seal between upper 244 and lower 246 regions.

In preferred embodiments, the above noted loading procedure may serve for initial intubation of a patient and/or for extubation. For initial intubation, the loading tube 530 is pre-loaded on the sleeve prior to intubation. The tube 260 is then lowered down into the trachea with the loading tube cover. When positioned at the desired location, the loading tube 530 is pulled out and thereby releasing the sleeve 220 to expand against the trachea wall.

For extubation, the loading tube 530 is lowered down around the breathing tube 260. The loading tube 530 thus comes to press on the wires 510 which in turn pull on and compress down the sleeve 220. Thereby, the loading tube 530 is encircling and confining the sleeve 220 within its lumen 535 to a tight configuration. Eventually the loading tube 530 is lowered along to preferably cover the full length of the sleeve 220. Then the intubation tube 260 may be pulled out of the trachea with the coving tube 530 on it, without the sleeve bruising the vocal cords or other sensitive tissue.

In preferred embodiments, the edge of the loading tube 530 is tapered in order to facilitate the scooping of the sleeve 220 into its lumen.

In preferred embodiments, the thickness of the loading tube 530 wall is less than 1 mm.

FIGS. 17a and 17b illustrate an alternative preferred embodiment of a sleeve loading mechanism, advantageously for encasing within a loading secondary tube.

At least one wire loop 1710 is threaded around and through the proximal top end of the sleeve 220. At least one wire extension 1720 is connected at one end to the loop 1710 and at the other end extending up towards the proximal end of the tube 260, and preferably outside the body of the patient.

In preferred embodiments, the wire 1720 is directed through a lumen within the ETT tube 260, as highlighted with a dashed line in FIG. 17a.

As illustrated in FIG. 17c, in practice the wire loop segment 1710 and wire extension segment 1720 may in fact be structured from a single physical wire in the form of a lasso shape, e.g., such that the wire loop 1710 has a ring 1730 at one end and is threaded through the ring 1730 at its other end, thus continue out as the extension 1720.

As illustrated in FIG. 17a, when the sleeve is in expanded state, the loop is open wide. When the proximal end of the wire 1720 is pulled then it exerts a pulling and closing force on the loop 1710, which in turn is exerting a pulling and closing force on the proximal end of the sleeve 220.

As illustrated in FIG. 17b, the wire extension 1720 can be pull up enough so that the loop 1710 and the proximal end of the sleeve 220 are brought tight around the ETT tube 260.

As illustrated in FIG. 17b, when the proximal end of the sleeve 220 is constricted closely around the ETT tube 260, an encasing secondary tube 530 can be lowered over the ETT, and eventually covering the sleeve similarly to as previously discussed with respect to FIG. 7b and FIG. 7c.

FIGS. 11A-11C relate to embodiments where, in order to radially inwardly movement sleeve 220 (i.e. against the outward bias of sleeve 220), it is advantageous to effect, on the distal half of sleeve 220 a force in the distal direction and/or to effect (for example, simultaneously) a force in the proximal direction on the proximal half of sleeve 220—for example, to ‘induce a longitudinal tension or ‘pulling force’ within sleeve 220 to force sleeve 220 to collapsed onto ETT tube 260.

As illustrated in FIG. 11A, in preferred embodiments there may be extensions or wires attachments 710 also on the distal end of the sleeve 220.

In some embodiments, the pulling force of elements 710 and/or 510 are substantially constant over the circumference (i.e. at the proximal or distal end of sleeve 210) of sleeve 210.

In preferred embodiments, the extensions or wires attachments 510 on the proximal end of the sleeve are attached at their other end to an inner ring 715. In preferred embodiments, said ring 715 (ie. situated on the proximal end of sleeve 220) may be pulled up and thereby via the attached extensions 510 exert a pulling force on the distal end of the sleeve.

As illustrated in FIG. 10A, in a preferred embodiment of the sleeve closing mechanism (i.e. to force the outwardly biased a single extension/wire attachment 710 can be linked to a wire loop 1010 which is threaded around close to the distal edge of the sleeve 220. In preferred embodiments the other end of wire 710 may be pulled from outside the patient body near the proximal end of the tube, and thereby a longitudinal pulling stretching force is exerted on the sleeve cylinder 220, particularly since the other end of the sleeve 220 is anchored by the extensions/wires 510 pulling resistance in the opposite direction. Such longitudinal pulling force has the physical result of tending to close the sleeve cylinder to a reduced diameter.

As illustrated in FIG. 10b, in alternative embodiments of the sleeve closing mechanism, one or more short extensions/wires 1020 extend from the sleeve cylinder 220 and have a loop or small rings 1030 at their ends. As shown in FIG. 10b, in some preferred embodiments more than one short extension 1020 may be joined to single small ring or loop 1030. A wire loop 1010 is going through said rings or loops 1020, and an extension/wire 710 is linked to said loop 1020. As discussed for a preceding embodiment, pulling on the wire 710 exerts a longitudinal pulling force on the sleeve, which has the physical result of tending to close the sleeve cylinder to a reduced diameter.

Thus, in FIG. 11B, wires 710 may be pulled in a distal direction (see the force of FIG. 11B). Thus, 710 is a ‘distal pulling element’. FIG. 710 shows an alternate structure for pulling a distal end of sleeve 220 in a distal direction. As illustrated in FIG. 11D, it is possible to provide a pivot point (or loop) which is fixed relative to ETT tube 260—upwards motion on a wire (or fiber—constructed of any appropriate material) near the proximal end of ETT tube 260 causes (because of pivot point 712) downward motion on distal pulling element 710.

As shown in FIG. 11A, there may be a plurality of such wires 710. In FIG. 11D, two particular wires 710X (synonymously ‘wire X’) and 710Y (synonymously ‘wire Y’) are configured so that upwards motion of 710X above sleeve 290 causes an upward force upon pivot point (or pivot element) 712 and increases a tension within wire/fiber 710 to cause a downwards force by wire 710 as illustrated in FIG. 11D—this may occur separately for wire X 710X and wire Y 710B.

In order to provide upwards force on proximal pulling element 510 it is not necessary to use a pivot point—it is possible just to provide wires or some other mechanism whereby the user may pull in a proximal direction to induce upwards force on/tension in proximal pulling element 510.

As with pulling element 510, distal element 710 may include a set of wires and/or a mesh or netting or any other appropriate physical structure for pulling in a distal direction on a distal end of sleeve 220.

### Technique for Assembling a Kit Comprising a Cuff Assembly and an ETT

FIG. 12 illustrates a technique for attaching a cuff assembly (including sleeve 220 and connecting element 290) to ETT tube 260. In the example of FIG. 12, the ETT tube is moved into a hole defined by the central void (e.g. see the ‘hole’ in the annularly shaped element of FIG. 14) of element 290. After the tube 260 passes through this void, it is possible for inward pressure of the material of connecting element 290 (e.g. silicone) to provide enough inward pressure upon tube 260 to hold cuff (i.e. including 220 and 290) to ETT tube 260.

For example, the inward pressure upon ETT tube 260 may be useful for retaining cuff assembly to ETT tube 260 such that the cuff remains in one place on the surface of ETT tube 260, without falling or sliding in a downward direction (i.e. due to the gravitational force on the cuff assembly due to the weight of the cuff assembly).

### A Discussion of an Apparatus of Method for Providing Suction in an Intubation System

Reference is now made to FIG. 13. As discussed earlier, the movement of conventional ETT cuff balloon within the trachea is a contributing factor to both leak of fluids and damage to the trachea tissue in presently-used ETTs. Due to head movement or tongue pressure, the upper portion of the tube is pushed and pulled upon by the patient at various times. In ETTs currently in use, this causes repeated movements of the cuff balloon along the trachea. The balloon thus rubs on the trachea tissue and slides over the lining fluids. Some embodiments herein are reduce such movement. Referring now to FIGS. 6a and 6b, a sleeve 220 is connected to ETT 260 via a thin, flexible membrane 290, which is attached, e.g. by gluing or ultrasonic welding, to both the ETT along an outer circumference thereof and to the sleeve along an inner circumference thereof. Because membrane 290 is easily deformable, in principle a significant freedom of up/down movement of the tube 260 relative to the trachea wall 210, without concomitant movement of sleeve 220, is enabled, depending on the length of membrane 290. Such relative movement is illustratively represented by the difference between FIGS. 4a and 4b, wherein the upward movement of the tube 260 has caused a change in the rest shape of the connecting membrane 290 but without dragging up the sleeve 220.

FIG. 13A-13B illustrates another preferred embodiment of the present invention. In particular, there is added a secondary suction port 675 below the connecting membrane 290 towards the distal end of the sleeve 220. Since patients commonly lay on their back, and thus fluids collect in the back side of the trachea, it is also preferred that the secondary port 675 will be located towards the back side of the sleeve. In preferred embodiments the suction of fluids via port 675 is operated via a separate lumen 670 extending from the port 675 up towards the proximal end of the ETT and out of the patient's body.

In order not to irritate the trachea tissue, it may be preferred that the port 675 location at the end of the lumen 670 will not extend beyond the edge of the sleeve 220. Yet, in order to have efficient suction of collected fluids, it is preferred that the port 675 will be close to the edge of the sleeve 220, e.g., within 5 mm, or 4 mm, or 3 mm, or 2 mm, or 1 mm from the edge of the sleeve 220.

Since the suction from said secondary port 675 is below the sealing connecting membrane element 290, it is influencing the air supply to the lungs. Therefore, in preferred embodiments of the invention, there will be particular correlation between the air ventilation phase and the suction of fluids via the port 675. For example, in preferred embodiments said suction will be performed during the exhaling (ventilator suction) of air out of the lungs. Conversely, in other preferred embodiments said suction will be performed during the inhaling (ventilator pushing) of air into of the lungs.

In yet other embodiments, the suction via port 675 will be at a continuous rate. In such case, the volume of air in ventilator action needs to be altered from present art in order to compensate for the suction volume via port 675. In particular, the suction port 675 air volume is subtracting from the ventilator inhalation of air supplied in via the tube 260 into the lungs and conversely adding to the ventilator suction out volume via the tube 260. Therefore common ventilation action is preferably modified in the following manner: the volume of air supplied in via the tube 260 need to be augmented to compensate for the loss out in suction by port 675, and conversely the volume of ventilator air sucked out via the tube 260 is reduced to take into account the parallel suckout action performed via port 675.

In some embodiments, upper suction port and/or lower suction port are respectively at the distal ends of respective suction tubes—for example, see the discussion of element 422 of FIG. 3A.

In other preferred embodiments, the wire mesh or braid 222 is only partially coated with impermeable coating layer 224, such that the coating is mainly around the area of the connecting membrane 290. In such a case, the port 675 is preferably located near the edge of the coating layer 224 in order not to irritate exposed tissue of the trachea wall.

### A Discussion of an Annularly-Shaped Connecting Element or Membrane According to Some Embodiments

FIG. 14 illustrates a substantially annularly shaped element (e, g. a membrane) 804 having an inner circumference 820 (which may or may not be circularly shaped) and an outer circumference 810 (which is typically substantially circularly shaped). In some embodiments, connecting element 290 may include one or more of the substantially annularly shaped elements of FIG. 14.

For both 804A and 804B, outer circumference 810 of element 804 is substantially circular. The inner portion may also be circular (see FIG. 14A—element 804A) having an inner radius r_{INNER} (or R^{ANNULUS}_{INNER}) or, alternatively (see FIG. have another shape (in the example of FIG. 14B, substantially triangular shaped with one or two or three rounded vertices)—in this case, it may be possible to discuss an ‘average inner radius’ AVG(r_{INNER}). Or AVG (R^{ANNULUS}_{INNER}). Preferably, the substantially annularly shaped element 810 is constructed of and/or coated with a material that is substantially impermeable to fluids.

FIG. 14 may be permanently attached to an interior of sleeve 220 so that the outer radius r_{OUTER} (or R^{ANNULUS}_{OUTER}) is equal to an inner radius of the sleeve element 220—different means of ‘permanent attachment’ are discussed above in what is not intended as an exhaustive list of techniques for permanent attachment. As noted in the discussion reference to FIG. 12, it may be desirable for element 290 (or at least a portion thereof at the inner circumference defining the inner void) to be constructed of an elastic material so that when ETT is deployed through the void, the inner circumference of element 810 will press in upon ETT 260 to directly attach element 810 (and to indirectly attach sleeve 220) to ETT 260.

In some embodiments, the presence of the ‘void’ of FIG. 14 facilitates the deploy the ETT through the ‘void.’

In an alternative embodiment, the ‘inner boundary’ or ‘circumference’ (not necessarily circular) 810 does not necessarily define the boundary of a void as depicted in FIGS. 14A-14B—instead, the ‘inner boundary’ may define the location where the connecting element 290 or membrane 290 includes ‘weaker’ material to define a ‘breaking point.’ (or predetermined breaking outline) through which a tube of any shape (for example, an ETT).

In one example, substantially all material within the ‘inner boundary’ 820 is ‘weaker material’ defining the break location throughout which the tube may be deployed (FIG. 8C). In another example, a ‘ring’ of material whose outer limit is defined by ‘inner boundary’ 820 is ‘weaker material’ defining the break location throughout which the tube may be deployed (FIG. 8D).

For the present disclosure, any statement referring to the ‘void’ (see, for example, FIGS. 8A-8B) may also refer to the region of ‘weaker material’ For example, statements referring to the average value of R(inner) (inner radius 820) and to the relationship between R(inner) (or R^{ANNULUS}_{INNER}) and R(outer) (or R^{ANNULUS}_{OUTER}).

### A Discussion of a Technique for Manufacturing a Cuff Assembly or a Portion Thereof

FIG. 15 illustrates a mold 908 for integral forming of the connecting membrane 290 and the sleeve coating 224 for some embodiments s whereby the connecting element 290 (e.g. annularly shaped as in 804 of FIG. 14) is constructed of the ‘coating material’ used to coat the fibrous element for sleeve 290. The mold 908 includes (i) a proximal portion 920 that is substantially cylindrically shaped (i.e. with a ‘large or outer mold radius); (ii) a distal portion 910 having substantially the same ‘large’ or outer mold radius which is substantially a radius of sleeve 290; (iii) a gap portion having a gap ‘thickness’ (which is at least 1% or 3% or at least 5% or least 10% and/or at most 30% or at most 20% and/or at most 15% and/or at most 10% a length of sleeve 290) and a gap ‘inner radius’ defined by inner piece 930 which is not necessarily cylindrical (i.e. it may be triangular in shape).

The combination of membrane 290 and sleeve (i.e. which may comprise wire skeleton 222 and coating 224) may be integrally manufactured by (i) first mounting the cylindrical wire skeleton 950 over the mold such that a mid-section of the wire skeleton 950 is overlapping with the gap between the mold cylinders 910, 920. Then it is possible to coating the mold (and also skeleton 950) (for example, by dipping the mold plus the skeleton 222 into the liquid coating) with the mounted wire simultaneously coat the wire skeleton. This may create a substantially liquid-impermeable layer (e.g. upon drying of the coating) over and between the wires of the skeleton 222 to simultaneously (i) create the coated sleeve 220 and, and (ii) create the substantially annularly-shaped membrane 804 (or connecting element 290 or a portion thereof) when the coating fills the gap between the cylinders (i.e. between proximal mold portion 920 and distal mold portion 910), while leaving a hole or void in the membrane (for example, corresponding to the void labeled and illustrated in FIG. 14) with the shape of the surface of the mold connector 930.

The thickness of the membrane 290 (or element 804) (i.e. in the ‘z’ or longitudinal direction’) may be set by the width of the gap between the left mold cylinder 910 (or ‘distal portion) and the right cylinder 920 (or ‘proximal portion).

The shape of the internal annular hole or void in the membrane may be set by the shape of the mold connector element 930 between the mold cylinders 910 and 920. The shape of the gap between the mold cylinders 910 and 920 in FIG. 8 is drawn to as flat, i.e., each of the cylinders as having a flat bottom shape, and consequently the resulting shape of the membrane. This is not meant to be limiting. To the contrary, in preferred embodiments it is desired that the cross section shape of the membrane 290 be concave (as illustrated for example in the cross section FIGS. 4a and 4b). Such non-flat membrane curvature is obtainable by the mold cylinders bottom/top surfaces being of a curved shape (e.g., a dome shape).

In the example of FIG. 4B, connecting portion 290 (which may be substantially annularly shaped as in 804 of FIG. 14) is located substantially at the midpoint (i.e. in a longitudinal direction) of sleeve 220. In the example of FIG. 4C, connecting portion 290 (which may be substantially annularly shaped as in 804 of FIG. 14) is located substantially at the distal end of sleeve 220. The ‘proximal distance’ between connecting element 290 (e.g. membrane—annularly shaped as in 804 of FIG. 14) and either the distal or proximal end of sleeve 220 may be regulated, during manufacture, by how skeleton 950 is deployed over mold 908—for example, if skeleton 950 is deployed so that the gap is near the distal end of skeleton 950, then after manufacture connecting element 290 may be near a distal end of sleeve 220 as in FIG. 4C. The case illustrated in FIG. 15, where skeleton 950 is placed over mold 908 so that the gap is substantially near a center of skeleton 950 corresponds more closely to the case of FIG. 4B, where connecting portion is near the center (i.e. in the longitudinal direction) of sleeve 220 (but not necessarily at the center).

Thus, it is possible to regulate the ‘longitudinal location’ of connecting element 290 and/or substantially annularly shaped element 804 (e.g. membrane) which will be, in the example of FIG. 15, integrally formed with a coating of sleeve 220 according to a relationship between (i) a location of either end of skeleton 950 as deployed on mold 908; and (ii) a gap location of ‘the gap.’

Some embodiments relate to methods for manufacturing a coated sleeve (e.g. with a fibrous skeleton) and to a product manufactured according to any method.

It will be appreciated that although the drawings depict the construction and use of an endotracheal tube, many embodiments of the present invention are equally applicable in the case of a tracheotomoty or tracheostomy tube, and are thus encompassed within the scope of this application.

Discussion of an Improved ETT Having an “Accordion” Section

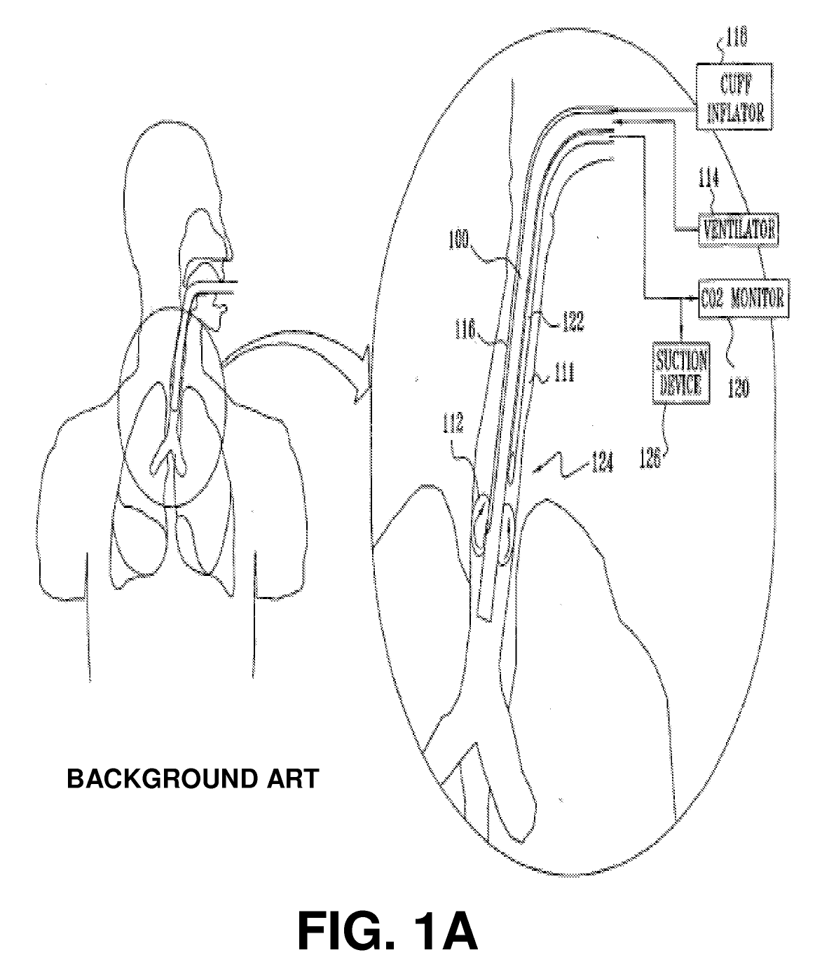

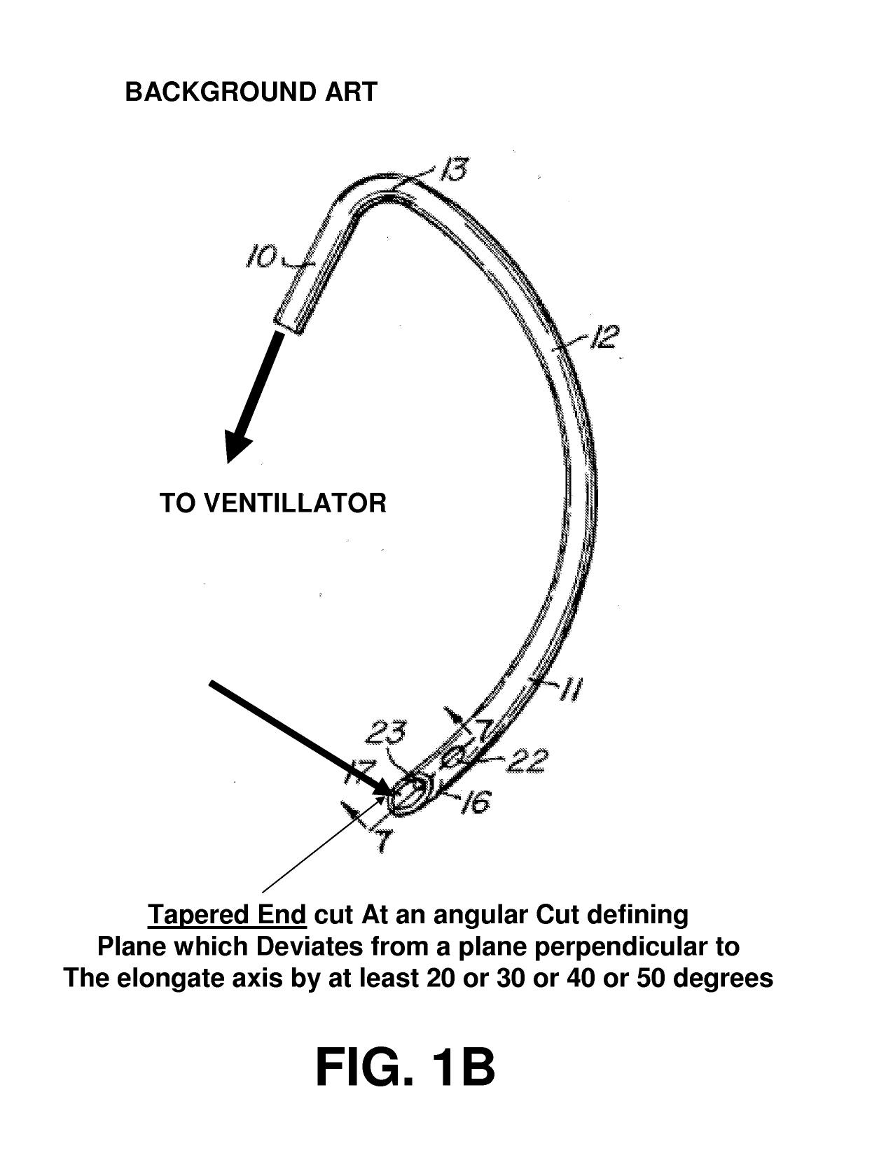

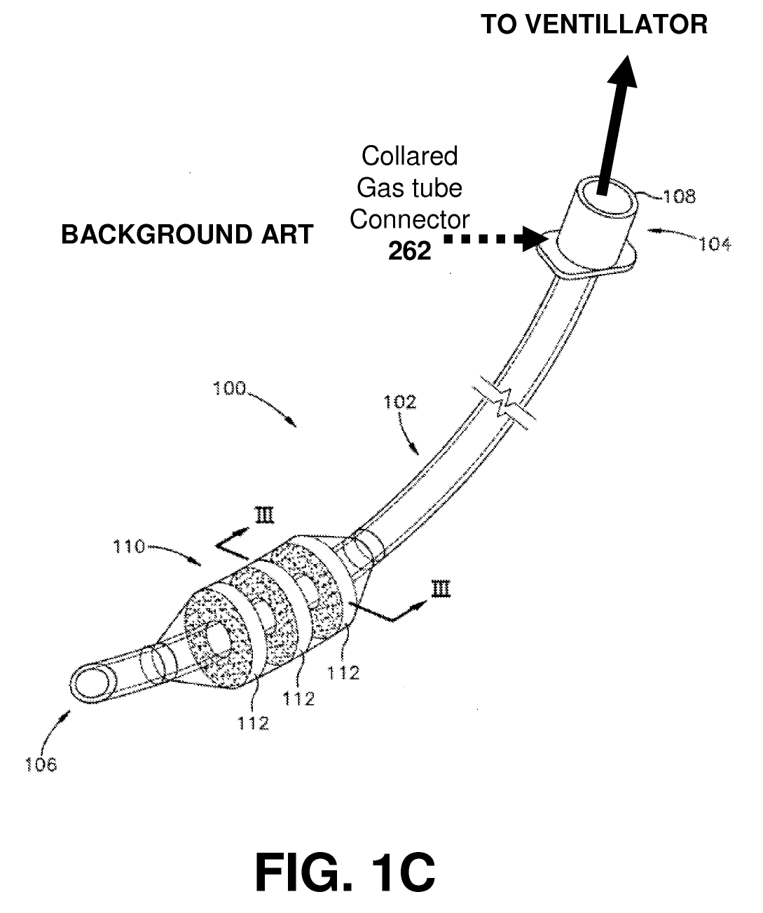

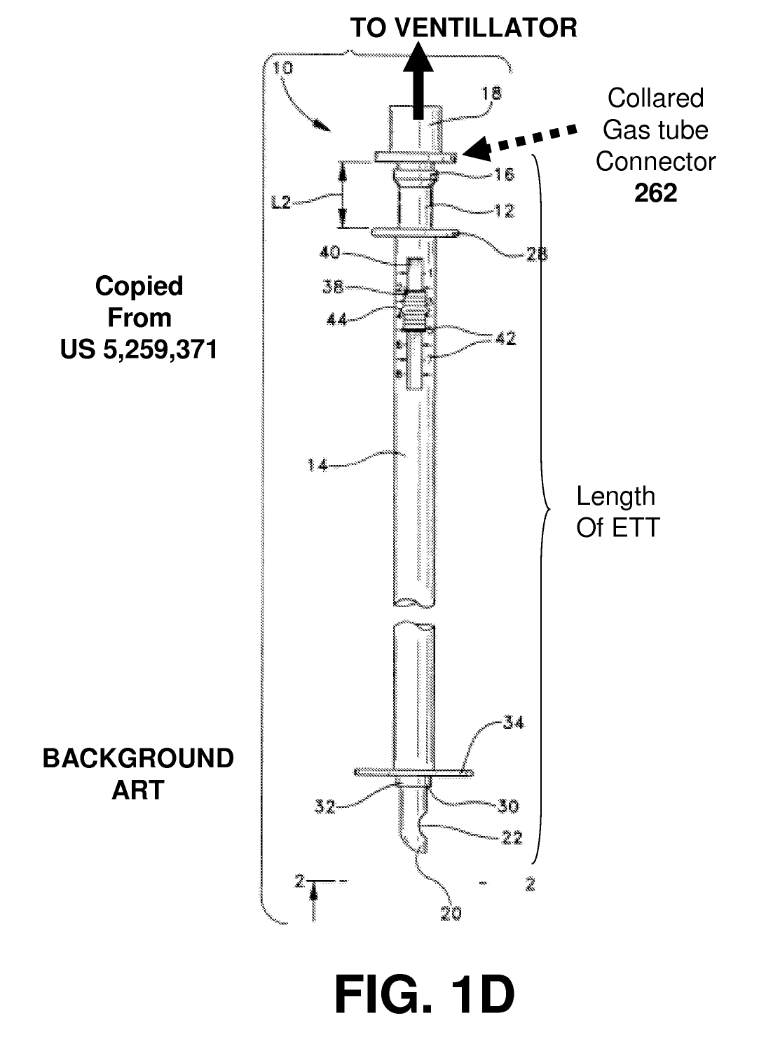

FIG. 16 refers to embodiments whereby one or more elastic elements are—see the related discussion in PCT/US2009/062227 filed on Oct. 27, 2009 incorporated herein by reference in its entirety. Thus, some embodiments relate to an apparatus for facilitating ETT intubation comprising: a) a ETT tube 260 (e.g. having a substantially uniform radius) having a length between 20 cm and 50 cm, a distal end of the ETT tube including a Murphy eye and/or a tapered end (see the comment on FIG. 1B), a majority of a length of the ETT tube being relatively stiff tubing, the ETT tube including one or more elastic sections 2165 (or 2175) deployed in a lower half of the ETT tube assembly, each elastic section having an equilibrium length between 0.5 and 2 cm and being longitudinally expandable by at least 50%. IN some embodiments, each elastic section is no closer than 4 cm from a closest neighboring elastic section via relatively stiff tubing. In some embodiments, an aggregate length of the elastic section(s) being at most 15% of a total length of the ETT tube.

In some embodiments, the apparatus further comprises: (i) a collared gas connector (see, for example, FIG. 1C) deployed to a proximal end of the ETT tube assembly.

### Discussion of Examples of a Fibrous Skeleton of an Outwardly-Biased Expandable Sleeve 220

In a first example, the fibrous skeleton may be constructed in the form of a mesh, or a braid, or a weave, or other textile form of assembling fiber into a tubular shape.

In another example, the fibrous skeleton may be constructed in the form of non-weaved net formed by molding into a cylindrical cast.

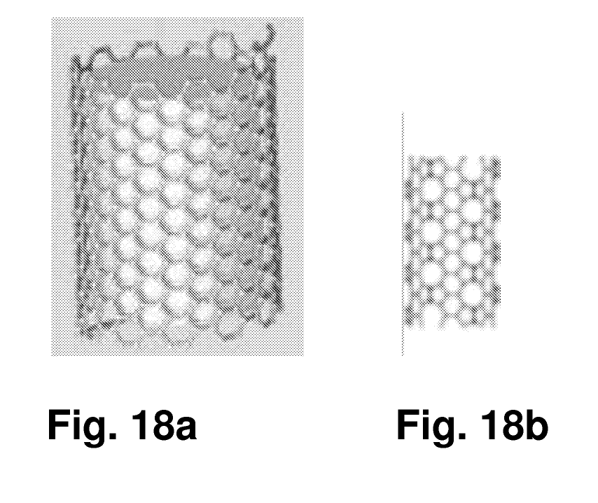

The “unit cell” in the previously noted figures were drawn to have the shape of a four sides polygon. Yet, this is not meant to be limiting. As illustrated in FIG. 18a, the shape of the net or mesh or braid unit cell may take other shapes such a honeycomb or even be non-uniform as illustrated in FIG. 18b.

The sleeve may also be constructed of a single molded material, such as by injection molding to the form of a cylindrical “honeycomb” or other cylindrical periodic arrangement of a net of cell walls with a membrane connecting the net walls. Thereby, the sleeve is constructed in a single molding step, including its cell walls and connecting membrane.

### A First Additional Discussion

In some embodiments, one or more or any combination of materials described herein are biocompatible for deployment into a trachea of a patient.

In some embodiments, zero or one or more of (i.e. any combination) of the following features may be provided:

a majority (i.e. by length—i.e. at least 50% or at least 60% or at least 70% or at least 80% or at least 90%) of a stent or stent-like sleeve 220 (i.e. a sleeve that includes a fibrous structure or skeleton coated with an elastic coating to be outwardly biased—for example, according to any property or combination of properties described in the present document) component surrounds a catheter (in this example, ETT tube 260); and/or
a majority (i.e. by length—i.e. at least 50% or at least 60% or at least 70% or at least 80% or at least 90%) of a stent or stent or stent-like sleeve 220 (i.e. a sleeve that includes a fibrous structure or skeleton coated with an elastic coating to be outwardly biased—for example, according to any property or combination of properties described in the present document) component surrounds an ETT tube (for example, having additional features in addition to just ‘catheter features’ the ETT tube includes additional features such as the distal-end Murphy eye and/or the tapered distal end (i.e. where instead of the tube ending at a ‘cut’ in a ‘perpendicular-plane’ that is perpendicular to the elongate axis, the end of the tube is cut at an ‘angular cut’ defined by a plane which deviates from the ‘perpendicular-plane’ by at least 20 or 30 or 40 or 50 or 60 degrees) and/or the collared gas line connector at the proximal end; and/or
Permanent attachment of a stent or stent-like sleeve 220 (i.e. a sleeve that includes a fibrous structure or skeleton coated with an elastic coating to be outwardly biased—for example, according to any property or combination of properties described in the present document) component to a catheter (for example, ETT) or to an ETT tube; and/or
A stent or stent-like sleeve 220 (i.e. a sleeve that includes a fibrous structure or skeleton coated with an elastic coating to be outwardly biased—for example, according to any property or combination of properties described in the present document) component facilitating blockage of a containing tube (for example, a biological lumen) in which it is deployed to create a longitudinal seal (e.g. together with one or more additional components including connecting element 290) between a proximal 244 and distal 246 regions within the containing tube (for example, a biological lumen); and/or
A membrane element (for embodiments where the connecting element includes a membrane element—for example, a thin membrane having a thickness less than 1 mm or less than 0.6 mm) attached at a substantially a mid-section (i.e. within a tolerance of 20% or 30% or 40% or 50% of a length of the stent-like element) of the stent-like element to the catheter; and/or
Attachment of a stent or stent-like sleeve 220 (i.e. a sleeve that includes a fibrous structure or skeleton coated with an elastic coating to be outwardly biased—for example, according to any property or combination of properties described in the present document) component to a catheter such as an ETT such that both ends of the stent or stent-like sleeve 200 remain free to expand (for example, the stent or stent-like sleeve is attached at a point that is not at the proximal or distal end—for example, separated from the proximal and distal end by at least 10% or 20% or 30% a length of the stent or stent-like sleeve); and/or
a stent or stent-like sleeve 220 (i.e. a sleeve that includes a fibrous structure or skeleton coated with an elastic coating to be outwardly biased—for example, according to any property or combination of properties described in the present document) component attached around a ETT where at least 10% or at least 20% or at least 30% or at least 40% by length of the stent longitudinally overlaps with the ETT tube.

### A Second Additional Discussion

A cuff assembly for facilitating tracheal intubation comprising a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein: i) according to the thin-wall feature, a ratio between a thickness of the sleeve and the expanded/equilibrium radius R_{EXPANDED} is at most 0.1; ii) according to the elasticity feature, when the sleeve is deployed within a rigid outer containing tube having a tube radius R_{TUBE} that is 0.8 times the fully-expanded radius R_{EXPANDED}, the sleeve exerts an outward pressure upon the outer containing tube whose value is at least 5 cm of water and at most 200 cm of water; and b) a membrane assembly 290 comprising at least one non-rigid at least partially obstructing membrane that is permanently attached to the sleeve, the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve, wherein the cuff assembly provides any one or both of a first feature and a second feature, the first and second features being defined as follows: according to the first feature, at least one fluid-communicating point 1037 on the inner surface of the sleeve that is located in the longitudinal interior region at a distance from both ends of the sleeve that is at least 10% of the sleeve length is not sealed from and/or is in fluid communication with at least one location on the outer surface of the sleeve; and according to the second feature, at least one of the membranes of the membrane assembly provides at least one of the following: i) an inner void at the defined longitudinal position, an average radius of the inner void having a value that is more than 0.5 mm and/or more than 0.02 times the expanded/equilibrium radius R_{EXPANDED} of the sleeve; and/or ii) a region of weakened membrane that has a material strength that is significantly weaker than a material strength of membrane material of the substantially annularly-shaped obstruction region such that when a blunt tube is pressed in a longitudinal direction through the region weakened membrane region, the inner void is formed.

In some embodiments, the membrane assembly substantially completely obstructs longitudinal fluid flow at the defined longitudinal position of the substantially annularly-shaped obstruction region.

In some embodiments, the membrane assembly is configured to allow fluid communication between first and second locations on the inner surface of the sleeve, the first location being at a proximal end of the sleeve and the second location being a distal end of the sleeve.

In some embodiments, in an average radius of the inner void is less than 7 mm.

A method comprising: effecting at least one longitudinal-traversing activity selecting from the group consisting of forcing a tube 260 through a cuff assembly and forcing a gas to flow longitudinally through the cuff assembly, the cuff assembly comprising a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein: i) according to the thin-wall feature, a ratio between a thickness of the sleeve and the expanded/equilibrium radius R_{EXPANDED} is at most 0.1; ii) according to the elasticity feature, when the sleeve is deployed within a rigid containing tube having a tube radius R_{TUBE} that is 0.8 times the fully-expanded radius R_{EXPANDED}, the sleeve exerts an outward pressure upon the outer tube whose value is at least 5 cm of water and at most 200 cm of water; and a membrane assembly 290 comprising at least one non-rigid at least partially obstructing membrane that is permanently attached to the sleeve, the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve, wherein: i) the tubing forcing is carried out such that the tube longitudinally traverses an interior of the cylindrical sleeve at a time that the at least one membrane is located in the interstitial region between the tube and the sleeve; and/or ii) the gas forcing causes the gas to flow longitudinally through the cuff assembly such that the forced gas longitudinally traverses an interior of the cylindrical sleeve and longitudinally traverses a region not occupied by the at least partially obstructing membrane that is permanently attached to the sleeve.

In some embodiments, the air forcing comprises over a period of time that is at least 60 minutes, causing the longitudinal air flow to change directions between a distal air flow direction and a proximal air flow direction, on average, at least 10 times per minutes.

In some embodiments, a maximum gas flow rate of the gas forcing is at least 50 cc per second.

A cuff assembly for facilitating tracheal intubation comprises: a) a substantially cylindrical sleeve 220 constructed from a fibrous skeleton that is coated with a coating so that the sleeve is substantially impermeable to liquids, a length of the sleeve being between 1 cm and 6 cm, the sleeve being radially expandable and outwardly biased to provide an expanded/equilibrium radius R_{EXPANDED} that is between 0.6 cm and 2 cm (in some embodiments, between 0.6 cm and 3 cm), and a fully-compressed radius R_{COMPRESSED} that is less than 80% of the expanded/equilibrium radius R_{EXPANDED} and/or that is at least 2 mm less than the fully-expanded radius R_{EXPANDED}; the sleeve providing elasticity properties so that when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 60 cm of water (for example, at most 50 cm of water), the sleeve including an inner surface and an outer surface; and b) a membrane assembly 290 comprising at least one non-rigid at least partially obstructing membrane that is permanently attached to the sleeve (for example, to the inner surface of sleeve) the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region contacting the inner surface of the sleeve at a defined longitudinal position, an inner radius and/or average inner radius of the obstruction region having a value that is less than 80% of the expanded/equilibrium radius R_{EXPANDED} of the sleeve, and wherein the cuff assembly provides any one or both of a first feature and a second feature, the first and second features being defined as follows: i) according to the first feature, at least one fluid-communicating point 1037 on the inner surface of the sleeve that is located in the longitudinal interior region at a distance from both ends of the sleeve that is at least 10% of the sleeve length is not sealed from and/or in fluid communication with at least one location on the outer surface of the sleeve; and ii) according to the second feature, the membrane provides at least one of: A) an inner void at the longitudinal position, an average radius of the inner void having a value that is more than 0.5 mm and/or more than 0.02 times the expanded/equilibrium radius R_{EXPANDED} of the sleeve; and/or B) a region of weakened membrane that has a material strength that is significantly weaker than a material strength of membrane material of the substantially annularly-shaped obstruction region such that when a tube is pressed in a longitudinal direction through the region weakened membrane region, the inner void is formed. In some embodiments, the membrane assembly substantially completely obstructs longitudinal fluid flow at the defined longitudinal position of the substantially annularly-shaped obstruction region.

In some embodiments, the membrane assembly is configured to allow fluid communication between first and second locations on the inner surface of the sleeve, the first location being at a proximal end of the sleeve and the second location being a distal end of the sleeve.

In some embodiments, an average radius of the inner void is less than 7 mm.

A method comprises

Forcing a tube 260 through an inner void of a cuff assembly and/or forcing liquid and/or gas to flow longitudinally through the inner void of the cuff assembly, the cuff assembly comprising: a) a substantially cylindrical sleeve constructed from a fibrous skeleton that is coated with a coating so that the sleeve is substantially impermeable to liquids, a length of the sleeve being between 1 cm and 6 cm, the sleeve being radially expandable and outwardly biased to provide an expanded/equilibrium radius R_{EXPANDED} that is between 0.6 cm and 3 cm, and a fully-compressed radius R_{COMPRESSED} that is less than 80% of the expanded/equilibrium radius R_{EXPANDED} and/or that is at least 2 mm less than the fully-expanded radius R_{EXPANDED}; the sleeve providing elasticity properties so that when the sleeve is deployed within a rigid tube having a tube radius R_{TUBE} that is 0.8 times the fully-expanded radius R_{EXPANDED}; the sleeve exerts an outward pressure upon the outer tube whose value is at least 5 cm of water and at most 50 cm of water, the sleeve including an inner surface and an outer surface; and

b) a membrane assembly comprising at least one non-rigid at least partially obstructing membrane that is permanently attached to the sleeve the at least one membrane being substantially impermeable to liquids, wherein the sleeve such that: the membrane assembly substantially obstructs longitudinal fluid flow in a substantially annularly-shaped obstruction region within the sleeve in a plane perpendicular to the central axis of the sleeve, the obstruction region contacting the inner surface of the sleeve at a defined longitudinal position, an inner radius and/or average inner radius of the obstruction region having a value that is less than 80% of the expanded/equilibrium radius R_{EXPANDED} of the sleeve.

In some embodiments, the rate of liquid and/or gas flow is on the order of magnitude of the rate of gas flow typically used in tracheal intubation (for example, at least 10% of this rate or at least 20% or this rate or at least 50% of this rate).

A cuff assembly for facilitating tracheal intubation comprises:

a) a substantially cylindrical sleeve constructed from a fibrous skeleton that is coated with a coating so that the sleeve is substantially impermeable to liquids, a length of the sleeve being between 1 cm and 6 cm, the sleeve being radially expandable and outwardly biased to provide an expanded/equilibrium radius R_{EXPANDED} that is between 0.6 cm and 2 cm (in some embodiments, between 0.6 cm and 3 cm), and a fully-compressed radius R_{COMPRESSED} that is less than 80% of the expanded/equilibrium radius R_{EXPANDED} and/or that is at least 2 mm less than the fully-expanded radius R_{EXPANDED}; the sleeve providing elasticity properties so that when the sleeve is deployed within a rigid tube having a tube radius R_{TUBE} that is 0.8 times the fully-expanded radius R_{EXPANDED}; the sleeve exerts an outward pressure upon the outer tube whose value is at least 5 cm of water and at most 60 cm of water (for example, at most 50 cm of water), the sleeve including an inner surface and an outer surface; and
b) a membrane assembly comprising at least one non-rigid at least partially obstructing membrane that is permanently attached to the sleeve; the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region contacting the inner surface of the sleeve at a defined longitudinal position, an inner radius and/or average inner radius of the obstruction region having a value that is less than 80% of the expanded/equilibrium radius R_{EXPANDED} of the sleeve,
c) at least one pulling element selected from the group consisting of:
i) a wire attached to the sleeve substantially at one end of the sleeve, a length of the wire being at least two twice the length of the sleeve; and/or
ii) a pulling element including an inner ring whose radius R^{PULLING}_{INNER}_{_{—}}_{RING} is between 0.2 and 0.8 times the expanded/equilibrium radius R_{EXPANDED}, the inner ring being connected to one end of the sleeve via an elongated element or a plurality of elongated elements such that the pulling element is permeable to fluids.
An intubation assembly comprises:
a) an ETT having a proximal and distal end, the ETT having length between 20 cm and 60 cm
b) a substantially cylindrical sleeve constructed from a fibrous skeleton that is coated with a coating so that the sleeve is substantially impermeable to liquids, a length of the sleeve being between 1 cm and 6 cm, the sleeve being radially expandable and outwardly biased to provide an expanded/equilibrium radius R_{EXPANDED} that is between 0.6 cm and 2 cm (in some embodiments, between 0.6 cm and 3 cm), and a fully-compressed radius R_{COMPRESSED} that is less than 80% of the expanded/equilibrium radius R_{EXPANDED} and/or that is at least 2 mm less than the fully-expanded radius R_{EXPANDED}; the sleeve providing elasticity properties so that when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 60 cm of water (for example, at most 50 cm of water), the sleeve including an inner surface and an outer surface, the ETT tube traversing the sleeve to define an interstitial region between the ETT and the sleeve; and
c) a membrane assembly comprising at least one non-rigid at least partially obstructing membrane that is permanently attached to the inner surface of the sleeve; the at least one membrane being substantially impermeable to liquids, the membrane assembly being attached to and/or in tight contact with an outer surface of the ETT tube to obstruct substantially all longitudinal flow in the interstitial region between (i) a proximal end of an outer surface of the ETT tube and (ii) a distal end of the outer surface of the ETT tube, wherein: (i) the sleeve and the membrane assembly are deployed so that a contact location between the membrane and the ETT is located in the distal or lower half of the ETT; and (ii) the membrane is permanently attached to the sleeve at a location that whose distance from a proximal end of the sleeve is at least 10% of a length of the sleeve.

In some embodiments, a membrane thickness is a membrane is at most 4 mm, or at most 2 mm, or at most 1 mm, or at most 0.5 mm.

In some embodiments, the inner radius and/or average inner radius of the obstruction region has a value that is less than 70% of the expanded/equilibrium radius R_{EXPANDED} of the sleeve or less than 60% of the expanded/equilibrium radius R_{EXPANDED} of the sleeve or less than 50% of the expanded/equilibrium radius R_{EXPANDED} of the sleeve or less than 40% of the expanded/equilibrium radius R_{EXPANDED} of the sleeve.

In some embodiments, the membrane assembly 290 comprising at least one non-rigid at least partially obstructing membrane is permanently attached to an inner surface of the sleeve.

In some embodiments, a distance between the fluid-communicating point 1037 on the inner surface of the sleeve and each end of the sleeve (i.e. both the proximal and distal end of the sleeve) is at least 10%, or at least 20%, or at least 30% or at least 40% of the sleeve length.

A cuff assembly for use with an ETT to facilitate tracheal intubation when the assembly is attached to the ETT, the cuff assembly comprising a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein: i) according to the thin-wall feature, a ratio between a thickness of the sleeve and the expanded/equilibrium radius R_{EXPANDED} is at most 0.1; ii) according to the elasticity feature, when the sleeve is deployed within a rigid outer containing tube having a tube radius R_{TUBE} that is 0.8 times the fully-expanded radius R_{EXPANDED}, the sleeve exerts an outward pressure upon the outer containing tube whose value is at least 5 cm of water and at most 200 cm of water; and b) a membrane assembly 290 comprising at least one non-rigid at least partially obstructing membrane that is permanently attached to the sleeve at a location on the inner surface of the sleeve at a location that is at or near mid-section of the sleeve and longitudinally removed from both the proximal and distal ends of the sleeve by a distance that is at least 20% a length of the sleeve, the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve.

A cuff assembly for use with an ETT to facilitate tracheal intubation when the assembly is attached to the ETT, the cuff assembly comprising a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein: i) according to the thin-wall feature, a ratio between a thickness of the sleeve and the expanded/equilibrium radius R_{EXPANDED} is at most 0.1; ii) according to the elasticity feature, when the sleeve is deployed within a rigid outer containing tube having a tube radius R_{TUBE} that is 0.8 times the fully-expanded radius R_{EXPANDED}, the sleeve exerts an outward pressure upon the outer containing tube whose value is at least 5 cm of water and at most 200 cm of water; and b) a membrane assembly 290 comprising at least one non-rigid at least partially obstructing membrane that is permanently attached to the sleeve at a location on the inner surface of the sleeve at a location that is at or near mid-section of the sleeve within a tolerance that is 20% a length of the sleeve, the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve.

A system for facilitating tracheal intubation comprising: a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein: i) according to the thin-wall feature, a ratio between a thickness of the sleeve and the expanded/equilibrium radius R_{EXPANDED} is at most 0.1; ii) according to the elasticity feature, when the sleeve is deployed within a rigid outer containing tube having a tube radius R_{TUBE} that is 0.8 times the fully-expanded radius R_{EXPANDED}, the sleeve exerts an outward pressure upon the outer containing tube whose value is at least 5 cm of water and at most 200 cm of water; and b) a membrane assembly 290 comprising at least one non-rigid at least partially obstructing membrane that is permanently attached to the sleeve, the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve; and c) a traversing tube 260 which longitudinally passes through the cylindrical sleeve to longitudinally traverse an interior of the sleeve, wherein the at least one membrane of the membrane assembly is sealingly in contact with an outer surface of the tube 260 to connect an outer surface of the tube 260 to an inner surface of the sleeve 220 via the at least one membrane 290.

An intubation system comprising: a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide an expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein: i) according to the thin-wall feature, a ratio between a thickness of the sleeve and the expanded/equilibrium radius R_{EXPANDED} is at most 0.1; ii) according to the elasticity feature, when the sleeve is deployed within a outer containing rigid tube having a tube radius R_{TUBE} that is 0.8 times the fully-expanded radius R_{EXPANDED}, the sleeve exerts an outward pressure upon the outer containing tube whose value is at least 5 cm of water and at most 200 cm of water; b) an ETT tube 260 having a proximal and distal end, the ETT longitudinally passing through an interior of the sleeve; c) a membrane assembly 290 comprising at least one non-rigid membrane that is permanently attached to the sleeve, the at least one membrane being substantially impermeable to liquids, the sleeve, the ETT tube and the membrane assembly being configured such that when the sleeve is deployed within a containing tube 210 so that the sleeve exerts an outward pressure upon a containing tube 210, the membrane assembly substantially longitudinally seals: i) an upper region 244 that is outside of tube 260, within the containing tube 210 and above the contacting non-rigid membrane 290 from ii) a lower region 246 that is outside of tube 260 within the containing tube 210, and below the contacting non-rigid membrane 290.

Some embodiments provide a cuff assembly for facilitating tracheal intubation comprising
a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein:
   i) according to the thin-wall feature, a ratio between a thickness of the sleeve and the expanded/equilibrium radius R_{EXPANDED} is at most 0.1;
   ii) according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 200 cm of water; and
b) a membrane assembly 290 comprising at least one non-rigid at least partially obstructing membrane that is permanently attached to the sleeve; the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve,
wherein the cuff assembly provides any one or both of a first feature and a second feature, the first and second features being defined as follows:
   according to the first feature, at least one fluid-communicating point 1037 on the inner surface of the sleeve that is located in the longitudinal interior region at a distance from both ends of the sleeve that is at least 10% of the sleeve length is not sealed from and/or is in fluid communication with at least one location on the outer surface of the sleeve; and
   according to the second feature, at least one of the membranes of the membrane assembly provides at least one of the following:
      i) an inner void at the defined longitudinal position, an average radius of the inner void having a value that is more than 0.5 mm and/or more than 0.02 times the expanded/equilibrium radius R_{EXPANDED} of the sleeve ; and/or
      ii) a region of weakened membrane that has a material strength that is significantly weaker than a material strength of membrane material of the substantially annularly-shaped obstruction region such that when a tube is pressed in a longitudinal direction through the region weakened membrane region, the inner void is formed.

In some embodiments, the membrane assembly substantially completely obstructs longitudinal fluid flow at the defined longitudinal position of the substantially annularly-shaped obstruction region.

In some embodiments, the membrane assembly is configured to allow fluid communication between first and second locations on the inner surface of the sleeve, the first location being at a proximal end of the sleeve and the second location being a distal end of the sleeve.

In some embodiments, an average radius of the inner void is less than 7 mm.

It is now disclosed a method comprising: effecting at least one longitudinal-traversing activity selecting from the group consisting of forcing a tube 260 through a cuff assembly and forcing a gas to flow longitudinally through the cuff assembly, the cuff assembly comprising
a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein:
   i) according to the thin-wall feature, a ratio between a thickness of the sleeve and the expanded/equilibrium radius R_{EXPANDED} is at most 0.1;
   ii) according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 200 cm of water; and
a membrane assembly 290 comprising at least one non-rigid at least partially obstructing membrane that is permanently attached to the sleeve; the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve,
wherein:
   i) the tubing force is carried out such that the tube longitudinally traverses the cylindrical sleeve at a time that the at least one membrane is located in the interstitial region between the tube and the sleeve;
   ii) the gas forcing causes the gas to flow longitudinally through the cuff assembly such that the forced gas longitudinally traverses the cylindrical sleeve and longitudinally traverses a region not occupied by material of the membrane within the at least partially obstructing membrane that is permanently attached to the sleeve.

In some embodiments, the air forcing comprises over a period of time that is at least 60 minutes, causing the longitudinal air flow to change directions by a distal air flow direction and a proximal air flow direction, on average, at least 10 times per minutes.

In some embodiments, a maximum gas flow rate of the gas forcing is at least 50 cc per second.

It is now disclosed a cuff assembly for facilitating tracheal intubation comprising:
a) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein:
   i) according to the thin-wall feature, a ratio between a thickness of the sleeve and the expanded/equilibrium radius R_{EXPANDED} is at most 0.1;
   ii) according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 200 cm of water; and
b) a membrane assembly 290 comprising at least one non-rigid at least partially obstructing membrane that is permanently attached to the sleeve; the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve, and
c) at least one pulling element selected from the group consisting of:
   a wire attached to the sleeve substantially at one end of the sleeve, a length of the wire being at least two twice the length of the sleeve; and/or an array of elongated connectors that are all attached to an inner ring whose radius^{PULLING}_{INNER}_{_{—}}_{RING} is between 0.2 and 0.8 times the expanded/equilibrium radius R_{EXPANDED}, the array of elongated connectors being distributed substantially over a circumference of the inner ring, the inner ring being connected to one end of the sleeve via an elongated element or a plurality of elongated elements such that the pulling element is permeable to fluids.

In some embodiments, the connectors are selected from the group including wires and elongated strips.

It is now disclosed an intubation system comprising:
a) an ETT having a proximal and distal end, the ETT having length between 20 cm and 60 cm,
b) a substantially cylindrical sleeve 220 constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide a expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein:
   i) according to the thin-wall feature, a ratio between a thickness of the sleeve and the expanded/equilibrium radius R_{EXPANDED} is at most 0.1;
   ii) according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 200 cm of water; and
c) a membrane assembly 290 comprising at least one non-rigid at least partially obstructing membrane that is permanently attached to the sleeve; the at least one membrane being substantially impermeable to liquids, wherein the sleeve and the at least one membrane are configured so that: the membrane assembly substantially obstructs longitudinal fluid flow in a substantially annularly-shaped obstruction region within the sleeve in a plane perpendicular to the central axis of the sleeve, the obstruction region delineated by the inner surface of the sleeve over the entire circumference of the inner surface of the sleeve at a defined longitudinal position, an inner radius and/or an average inner radius of the obstruction region having a value that is less than 80% of the expanded/equilibrium radius R_{EXPANDED} of the sleeve, the membrane assembly being attached to and/or in tight contact with an outer surface of the ETT tube to obstruct substantially all longitudinal flow in the interstitial region between (i) a proximal end of an outer surface of the ETT tube and (ii) a distal end of the outer surface of the ETT tube, wherein:
   i) the sleeve and the membrane assembly are deployed so that a contact location between the membrane and the ETT is located in the distal or lower half of the ETT; and
   ii) the membrane is permanently attached to the sleeve at a location that whose distance from a proximal end of the sleeve is at least 10% of a length of the sleeve.
      In some embodiments, the system further comprises:
d) a loading tube 530 having a radius R^{LOADING}_{TUBE} that is substantially equal to the radius of the ETT tube R^{ETT}_{TUBE} within a tolerance of 20%, the loading tube positioned so that at least a portion the sleeve is compressed within the loading tube between the ETT tube and the loading tube 530.

In some embodiments, a majority of the sleeve is compressed within the loading tube between the ETT tube and the loading tube 530.

In some embodiments, substantially an entirety of the sleeve is compressed within the loading tube between the ETT tube and the loading tube 530.

It is now disclosed a method of preparing an ETT device comprising:

a) at a time that an ETT traverses a sleeve that is radially expandable and outwardly biased, and at a time when the ETT is attached to the sleeve by at least one pulling element connecting a proximal end of the sleeve to the ETT, sliding a loading tube 530 in a distal direction towards the sleeve, a radius of the loading tube R^{LOADING}_{TUBE} being substantially equal to the radius of the ETT tube R^{ETT}_{TUBE} within a tolerance of 20%; and

b) causing at least a portion of the radially expandable sleeve to decrease its radius to a value that is less than equal to the radius of the loading tube R^{LOADING}_{TUBE} and greater than or equal to the radius of the ETT; and

c) sliding the loading tube in a distal direction so that at least a majority of the sleeve is positioned within the loading tube.

In some embodiments, step (b) is carried out before step (c).

In some embodiments, step (b) includes pulling a wire connecting a proximal end of the sleeve with the ETT in a proximal direction so that tension in the pulled wire induces an inward force on a proximal end of the sleeve, thereby causing the at least a portion of the radially expandable sleeve to decrease its radius tightly around the ETT tube.

In some embodiments, step (b) is carried out by distal motion of the loading tube

In some embodiments, the distal motion of the loading tube causes the loading tube to engage a pulling element connecting a proximal end of the sleeve to the ETT, thereby increasing tension within the pulling element, thereby causing the at least a portion of the radially expandable sleeve to decrease its radius.

It is now disclosed a method of modifying a structure of an ETT cuff, the method comprising:

at a time that:
i) an ETT traverses a sleeve that is radially expandable and outwardly biased;
ii) the sleeve radius is substantially equal to an expanded/equilibrium radius R_{EXPANDED}; and
iii) the sleeve is attached to the ETT at a proximal end of the sleeve by one or more proximal pulling elements and the sleeve is attached to the ETT at a distal of the sleeve by one or more distal pulling element,
inducing tension within the pulling elements to simultaneously cause a proximal tension to prevail in the proximal pulling element(s) and a distal tension to prevail in the distal pulling element(s), the induced tensions within the pulling elements serving to:
   i) induce a longitudinal tension in the sleeve to longitudinally stretch the sleeve; and
   ii) cause the sleeve radius to decrease by a factor of at least 30%

so that the sleeve edge inner radius is substantially equal to the outer ETT radius within a tolerance of 10%.

In some embodiments, the tension in the proximal pulling elements is induced by distal motion of a loading tube.

In some embodiments, the tension in the proximal pulling elements and/or in the distal pulling elements is induced by pulling a wire connecting the sleeve to the ETT in a proximal direction, the wire having a length that is at least twice the length of the sleeve.

In some embodiments, the apparatus further comprises:

A first suction tube 422A having a suction tube radius that is at least 50% of a radius of the ETT, a length of the first suction tube being at least 40% of a length of the ETT, a proximal end of the first suction tube being located proximally relative to a proximal end of the sleeve, a distal end of the first suction port forming a lower suction port 675 that is located below a membrane of the membrane assembly that is permanently attached to the sleeve and attached to and/or in tight contact with the outer surface of the ETT tube.

In some embodiments, the lower suction port 675 is located substantially at or near the inner surface of the sleeve.

In some embodiments, the lower suction port 675 is located substantially at the distal end of the sleeve.

In some embodiments, the apparatus further comprises;

a second suction tube 422B having a suction tube radius that is at least 50% of a radius of the ETT, a length of the second suction tube being at least 40% of a length of the ETT, a proximal end of the first suction tube being located proximally relative to a proximal end of the sleeve, a distal end of the first suction port forming an upper suction port 420 that is located above a membrane of the membrane assembly that is permanently attached to the sleeve and attached to and/or in tight contact with the outer surface of the ETT tube.

In some embodiments, the radius of the first and/or second suction tubes is at most 30% of the radius of the ETT.

In some embodiments, the biocompatible coating of the cylindrical sleeve includes one or more of silicone, polyurethane and latex.

In some embodiments, a membrane thickness is a membrane is at most 4 mm, or at most 2 mm, or at most 1 mm, or at most 0.5 mm.

In some embodiments, the inner radius and/or average inner radius of the obstruction region has a value that is less than 70% of the expanded/equilibrium radius R_{EXPANDED} of the sleeve or less than 60% of the expanded/equilibrium radius R_{EXPANDED} of the sleeve or less than 50% of the expanded/equilibrium radius R_{EXPANDED} of the sleeve or less than 40% of the expanded/equilibrium radius R_{EXPANDED} of the sleeve.

In some embodiments, the membrane assembly 290 comprising at least one non-rigid at least partially obstructing membrane is permanently attached to an inner surface of the sleeve.

In some embodiments, a distance between the fluid-communicating point 1037 on the inner surface of the sleeve and each end of the sleeve (i.e. both the proximal and distal end of the sleeve) is at least 10%, or at least 20%, or at least 30% or at least 40% of the sleeve length.

In some embodiments, the length of the sleeve is between 2 cm and 6 cm.

In some embodiments, a value of radius the expanded/equilibrium radius R_{EXPANDED} is between 1 cm and 1.7 cm.

In some embodiments, according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 100 cm of water.

In some embodiments, according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 60 cm of water.

In some embodiments, according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 40 cm of water.

In some embodiments, according to the elasticity feature, when the sleeve is deployed within a rigid tube having a tube radius R_{TUBE} that is 0.8 times the fully-expanded radius R_{EXPANDED}, the sleeve exerts an outward pressure upon the outer tube whose value is at least 5 cm of water and at most 25 cm of water.

In some embodiments, a centroid of the region of weakened membrane is substantially at a location of the center of the membrane in which it resides.

In some embodiments, the sleeve provides both the thin wall feature and the elasticity feature.

In some embodiments, the sleeve provides only the thin wall feature.

In some embodiments, the sleeve provides only the elasticity feature.

In some embodiments, the compressed radius R_{COMPRESSED} that is less than 50% of the expanded/equilibrium radius R_{EXPANDED}

In some embodiments, the compressed radius R_{COMPRESSED} that is less than 40% of the expanded/equilibrium radius R_{EXPANDED}

In some embodiments, the compressed radius R_{COMPRESSED} that is less than 30% of the expanded/equilibrium radius R_{EXPANDED}

It is now disclosed an apparatus for facilitating ETT intubation comprising: a) a ETT tube 260 (e.g. having a substantially uniform radius) having a length between 20 cm and 50 cm, a distal end of the ETT tube including a Murphy eye and/or a tapered end, a majority of a length of the ETT tube being relatively stiff tubing, the ETT tube including one or more elastic sections 2165 deployed in a lower half of the ETT tube assembly, each elastic section having an equilibrium length between 0.5 and 2 cm and being longitudinally expandable by at least 30%.

In some embodiments, the elastic section is longitudinally expandable by at least 50%.

In some embodiments, each elastic section is no closer than 4 cm from a closest neighboring elastic section via relatively stiff tubing.

In some embodiments, an aggregate length of the elastic section(s) being at most 15% of a total length of the ETT tube.

It is now disclosed for the first time a cuff assembly (for example, the combination of elements 290 and 220 of FIG. 3B—for example, element 290 may have one or more properties of element 804 of FIG. 14) for facilitating tracheal intubation comprising: a substantially cylindrical sleeve 290 (for example, having stent-like properties); and (ii) at least one non-rigid at least partially obstructing annulus 804 and/or at least one substantially annularly shaped membrane 804 including a substantially circular outer circumference and a (not necessarily circular) void located at or near the center of the annulus and/or substantially annularly shaped membrane. The cylindrical sleeve may be constructed from a fibrous skeleton or fibrous structure that is coated along at least a portion of its length with a coating of elastic material so that said portion of the sleeve is substantially impermeable to liquids, said portion being more that 20% or 30% or 50% or 70% or 90% or 100% of length of the fibrous skeleton; a length of the sleeve being at least 1 cm (for example, at least 2 cm or at least 3 cm) and/or most 20 cm and/or at most 15 cm and/or at most 10 cm and/or at most 6 cm (for example, between 1 cm and 6 cm) (this may refer to either the total length of the sleeve or the length of the coated portion of the sleeve), the sleeve being radially expandable and outwardly biased to provide an expanded/equilibrium radius R_{EXPANDED} (i.e. the radius when is allowed to expand to its equilibrium radius in the absence or external forces) that is between 0.4 cm and 2 cm (in some embodiments, between 1 cm and 1.5 cm), and capable of being compressed to a compressed radius R_{COMPRESSED} that is less than 80% (in some embodiments, less than 70% or less than 60% or less than 50%) of the expanded/equilibrium radius R_{EXPANDED} and/or to a compressed radius that is at least 2 mm (in some embodiments, at least 3 mm or at least 4 mm or at least 5 mm) less than the fully-expanded radius R_{EXPANDED} of the sleeve.

The cylindrical sleeve 290 may provide elasticity properties so that when the sleeve is deployed within a rigid tube having a tube radius R_{TUBE} that is less than the expanded/equilibrium radius R_{EXPANDED} (for example, the tube radius R_{TUBE} is between 0.7 and 0.9 times (for example, 0.8 times) the expanded/equilibrium radius R_{EXPANDED}) the sleeve exerts an outward pressure upon the outer tube whose value is at least 5 cm of water and at most 50 cm of water and/or between 0.5 kPA (kilopascals) and 5 kPA.

As noted above, the cuff assembly (i.e. including at least sleeve 220 and element 290—for example, similar to element 804 of FIG. 14) may include at least one non-rigid obstructing annulus 804 and/or at least one substantially annularly shaped membrane 804 and/or at least one membrane with a substantially circular outer perimeter. This at least one non-rigid obstructing annulus and/or at least one substantially annularly shaped membrane and/or membrane having a circular outer perimeter which is permanently attached to the sleeve 290 at a location that is removed from the proximal end of the sleeve 290 by at least 3 mm (or at least 5 mm or 10 mm or 15 mm or 20 mm) and/or at least 5% or at least 10% or 30% or 50% or 70% of a length of the sleeve (i.e. either the entire length of the sleeve or the length of the coated portion).

In some embodiments, the annulus and/or substantially annularly shaped membrane 804 (for example, see being substantially impermeable to liquids) has an average outer radius R^{ANNULUS}_{OUTER} that is equal to at least 0.8 times (or at least 0.7 or 0.9 or 1.0 or 1.1 or 1.2) the expanded/equilibrium radius of the sleeve. In some embodiments, the annulus and/or substantially annularly shaped membrane 804 has an average inner radius R^{ANNULUS}_{INNER} whose value is at least 2 mm (or at least 3 mm or at least 4 mm at least 5 mm) less than the average outer radius R_{R}^{ANNULUS}_{OUTER} and/or whose value is at most 80% (or at most 70% or at most 60%) of the average outer radius R^{ANNULUS}_{OUTER}.

In a first example, the fibrous skeleton may be constructed in the form of a mesh, or a braid, or a weave, or other textile form of assembling fiber into a tubular shape.

In another example, the fibrous skeleton may be constructed in the form of non-weaved net formed by molding into a cylindrical cast.

The “unit cell” in the previously noted figures were drawn to have the shape of a four sides polygon. Yet, this is not meant to be limiting. As illustrated in FIG. 18a, the shape of the net or mesh or braid unit cell may take other shapes such a honeycomb or even be non-uniform as illustrated in FIG. 18b.

The sleeve may also be constructed of a single molded material, such as by injection molding to the form of a cylindrical “honeycomb” or other cylindrical periodic arrangement of a net of cell walls with a membrane connecting the net walls. Thereby, the sleeve is constructed in a single molding step, including its cell walls and connecting membrane.

It is now disclosed for the first time a method of assembly of an intubation device comprising:

(a) providing any cuff assembly of disclosed herein (for example, the cuff assembly described above) (b) providing a hollow ETT tube 260 having a length between 20 and 50 cm and an ETT tube radius R^{ETT}_{TUBE} that is:
substantially equal or larger than the inner radius of the annulus R^{ANNULUS}_{INNER}; and/or
at least 2 mm less than the outer radius; and/or
at most 80% of the outer radius R^{ANNULUS}_{OUTER}
(c) inserting the ETT tube 260 through the sleeve 220 and the annulus 290 (or 804) so that inward pressure of the annulus 804 upon the ETT tube 260 retains the annulus and the sleeve upon the ETT tube so that a location of a midpoint of the sleeve and/or location of a ‘contact point’ 258 between an inner surface of the annular membrane 290 and ETT is located within the bottom ⅓ or bottom ½ of the ETT tube and optionally a minimum distance from the bottom of the ETT tube—minimum distance is 1 cm or 2 cm or 3% or 5% etc from the bottom of the tube. The method may be performed so that after deployment of the cuff assembly including the connecting element or membrane 290 and the sleeve 220 onto the ETT tube 260 a seal is formed between (i) a proximal end of the outer surface of the ETT tube and (iii) a distal end of the outer surface of the ETT tube.

In some embodiments, the method may be performed so that the cuff assembly including the connecting element or membrane 290 and the sleeve 220 is attached to ETT 260 in a manner so that cuff assembly including the connecting element or membrane 290 and the sleeve 220 is ‘reversibly attached’ to the ETT 260 and is thus ‘reversibly attachable.’

It is now disclosed a system comprising:
any cuff assembly of disclosed herein (for example, the cuff assembly described above; and (b) a hollow ETT tube 260 having a length between 20 and 50 cm and a radius that is:
substantially equal or larger than the inner radius of the annulus R^{ANNULUS}_{INNER} within a tolerance of 15%; and/or
at least 2 mm less than the outer radius; and/or
at most 80% of the outer radius R^{ANNULUS}_{OUTER}
the cuff assembly being deployed upon the ETT tube so that inward pressure of the annulus upon the ETT tube by the annulus retains the annulus and the sleeve upon the ETT tube a location near an end of the ETT tube
In some embodiments, the system further comprises:
(c) a loading or ‘encasing’ tube 530 having a loading tube radius R^{LOADING}_{TUBE} that is substantially equal to the radius of the ETT tube R^{ETT}_{TUBE} within a tolerance of 20% (or 10% or 30% or 40% or 50%) the loading tube positioned so that substantially an entirety of the sleeve is compressed within the loading tube between the ETT tube and the loading tube 530.

In some embodiments, the system further comprises: a pulling element 510 having a distal end attached to a proximal end of the sleeve and an inner ring whose radius R^{PULLING}_{INNER}_{_{—}}_{RING} is equal (i.e. within a tolerance of 50% or 40% or 30% or 20% or 10%) to the ETT radius and/or the average inner radius of the annulus, the pulling element being permeable to fluids. In some embodiments, the pulling element is oriented ‘upwardly’ so that an angle:

a line segment between the proximal end of the sleeve and a location on the inner ring; and
a central axis of the sleeve is at most is less than 90 degrees.

In some embodiments, the pulling element is constructed from a fibrous net and/or an array of wires and/or an array of strips—for example, the pulling element may be constructed of fibers of the fibrous structure (or skeleton)—i.e. integrally formed with the skeleton (but not necessarily covered with the impermeable coating).

In some embodiments, portion of the inner region of said annulus is glued or welded to the outer surface of said ETT tube.

Some embodiments of the present invention provide related kits.

It is now disclosed for the first time a system for tracheal intubation in a patient in need thereof, comprising, an air passage tracheal tube having a distal end which is inserted into the trachea and a proximal end which remains outside the trachea, and a cuff element placed towards the distal end of said tracheal tube;

where said cuff element is comprised of a sleeve through which said tracheal tube passes and a connecting structure between said sleeve and said tracheal tube;
said sleeve having an inner surface which faces said tracheal tube and an outer surface and proximal and distal ends which correspond to the proximal and distal ends, respectively, of said tracheal tube, the sleeve being of a length less than the distance between the larynx and the carina; and
wherein the sleeve is a self-expanding sleeve, biased toward an expanded state, which in the fully self-expanded state is of an inner diameter larger than the outer diameter of said tracheal tube by at least a difference of 2 mm, and which in the fully self-expanded state the sleeve outer diameter is larger than the diameter of the target human trachea lumen yet smaller than twice the target human trachea lumen diameter; and
where said connecting structure which contacts at least a portion of the outer surface of said tracheal tube and is permanently attached to at least a portion of said inner surface of said sleeve in a manner which substantially creates a seal between the distal and proximal ends of the inner surface of said sleeve and the distal and proximal ends of the outer surface of said tracheal tube. In some embodiments, the sleeve comprises of a wire mesh or braid skeleton which is covered by one or more elastic covering layers which are impermeable to mucous, saliva and other bodily fluids.

In some embodiments, said elastic covering layer is made of a material selected from: silicon, latex, or polyurethane.

In some embodiments, said connecting structure is a flexible membrane, said flexible membrane is generally disk-, ring- or cone-shaped; wherein along at least an inner circumference of the membrane the membrane tightly contacts the tube and along the outer circumference of the membrane the membrane contacts the inner surface of the sleeve.

In some embodiments, said membrane is attached to the sleeve by, molding together, or gluing, or ultrasonic welding.

In some embodiments, the connecting structure is an inflatable balloon, the length of which is less than the length of said sleeve, and wherein the balloon is attached to the sleeve by, molding together, or gluing, or ultrasonic welding.

In some embodiments, where two or more elongated wire-like or band-like extensions (i.e. a pulling element 510) are linked at one end to a portion of the proximal end of said sleeve, and said extensions are linked at their other end to said tracheal tube.

In some embodiments, extensions (of element 510—FIG. 7A) comprise of wires made of the same material as said sleeve skeleton mesh.

In some embodiment, said extensions (of element 510—FIG. 7A) comprise of elastic bands made of the same material as said sleeve elastic covering layer.

In some embodiments, the system including no inflatable elements and no inflating lumen and/or does not rely on an inflatable element or lumen to provide outward pressure on the trachea.

In some embodiments, the system present only two lumens extending out to the distal end; the ETT breathing tube and the suction tube, and including no inflating tube.

In some embodiments, the system further comprises suction ports whereat least one first suction port is located above and at least one second suction port is located below said membrane towards the distal end of said sleeve.

In some embodiments, the system further comprises a lumen connecting from said second suction port to a suction machine located outside of the body of said patient.

In some embodiments, the system further the suction port is located at a distance from the distal end of said sleeve of where said distance is selected to be less than 5 mm, or less than 4 mm, or less than 3 mm, or less than 2 mm, or less than 1 mm.

Various embodiments of the invention have been described in detail, but it will be understood by those skilled in the art that variations and modifications can be effected within the spirit and scope of the invention.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the general combination of parts that perform the same functions as exemplified in the embodiments, and includes both combinations and sub-combinations of the various features described hereinabove as well as variations and modifications thereof, which would occur to persons skilled in the art upon reading the foregoing description.

Having thus described the foregoing exemplary embodiments it will be apparent to those skilled in the art that various equivalents, alterations, modifications, and improvements thereof are possible without departing from the scope and spirit of the claims as hereafter recited. In particular, different embodiments may include combinations of features other than those described herein. Accordingly, the claims are not limited to the foregoing discussion.

1-67. (canceled)

68. An intubation system comprising:
a) a substantially cylindrical sleeve constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide an expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein:
i) according to the thin-wall feature, a ratio between a thickness of the sleeve and the expanded/equilibrium radius R_{EXPANDED} is at most 0.1;
ii) according to the elasticity feature, when the sleeve is deployed within a outer containing rigid tube having a tube radius R_{TUBE} that is 0.8 times the fully-expanded radius R_{EXPANDED}, the sleeve exerts an outward pressure upon the outer containing tube whose value is at least 5 cm of water and at most 200 cm of water;
b) an ETT tube having a proximal and distal end, the ETT longitudinally passing through the sleeve;
c) a membrane assembly comprising at least one non-rigid membrane that is permanently attached to the sleeve, the at least one membrane being substantially impermeable to liquids, the attached non-rigid membrane being sealingly in contact with an outer surface of the ETT tube to create a substantial liquid-tight sleeve within the sleeve , thereby sealing an upper region that is outside of tube within the sleeve and above the contacting non-rigid membrane from a lower region that is outside of tube , within the sleeve and below the contacting non-rigid membrane .

69. The intubation system of claim 68 wherein the sleeve and the membrane assembly are deployed so that a contact location between the membrane and the ETT is located in the distal or lower half of the ETT.

70. The intubation system of claim 68 wherein the membrane is permanently attached to the sleeve at a location that whose distance from a proximal end of the sleeve is at least 10% of a length of the sleeve.

71. The intubation system of claim 68, wherein the membrane is sealingly in contact with an outer surface of the ETT tube by means of elastic pressure and/or by means of glue and/or by means of welding.

72. The intubation system of claim 68 further comprising:
d) a loading tube having a radius R^{LOADING}_{TUBE} that is substantially equal to the radius of the ETT tube R^{ETT}_{TUBE} within a tolerance of 50%, the loading tube positioned so that at least a portion the sleeve is compressed within the loading tube between the ETT tube and the loading tube .

73. The intubation system of claim 72 wherein a majority of the sleeve is compressed within the loading tube between the ETT tube and the loading tube .

74. The system of claim 72 wherein substantially an entirety of the sleeve is compressed within the loading tube between the ETT tube and the loading tube .

75. The system of claim 68 further comprising:
d) a first suction tube having a suction tube radius that is less than 50% of a radius of the ETT, a length of the first suction tube being at least 40% of a length of the ETT, a proximal end of the first suction tube being located proximally relative to a proximal end of the sleeve, a distal end of the first suction port forming a lower suction port that is located below a membrane of the membrane assembly that is permanently attached to the sleeve and attached to and/or in tight contact with the outer surface of the ETT tube.

76. The system of claim 75 wherein the lower suction port is located substantially at or near the inner surface of the sleeve.

77. The system of claim 75 wherein the lower suction port is located substantially at the distal end of the sleeve.

78. The system of claim 75 further comprising:
e) a second suction tube having a suction tube radius that is less than 50% of a radius of the ETT, a length of the second suction tube being at least 40% of a length of the ETT, a proximal end of the first suction tube being located proximally relative to a proximal end of the sleeve, a distal end of the first suction port forming an upper suction port that is located above a membrane of the membrane assembly that is permanently attached to the sleeve and attached to and/or in tight contact with the outer surface of the ETT tube.

79. The system of claim 75 wherein the radius of the first and/or second suction tubes is at most 30% of the radius of the ETT.

80. An apparatus for facilitating ETT intubation comprising: a) a ETT tube , a distal end of the ETT tube including a Murphy eye and/or a tapered end, a majority of a length of the ETT tube being relatively stiff tubing, the ETT tube including one or more elastic sections deployed in a lower half of the ETT tube assembly, each elastic section having an equilibrium length between 0.5 and 2 cm and being longitudinally expandable by at least 30%.

81. The apparatus of claim 80 wherein the elastic section is longitudinally expandable by at least 50%.

82. The apparatus of claim 80 wherein each elastic section is no closer than 4 cm from a closest neighboring elastic section via relatively stiff tubing.

83. The apparatus of claim 80 wherein an aggregate length of the elastic section(s) is at most 15% of a total length of the ETT tube.

84. An intubation system comprising:
a) a substantially cylindrical sleeve constructed from a fibrous skeleton that is coated with a biocompatible elastic coating so that the sleeve is substantially impermeable to liquids, the sleeve including an inner surface and an outer surface, a length of the sleeve being between 1 cm and 8 cm, the sleeve being radially expandable and outwardly biased to provide an expanded/equilibrium radius R_{EXPANDED} that is between 0.6 cm and 2.2 cm, the sleeve being compressible to a compressed radius R_{COMPRESSED} that is less than 60% of the expanded/equilibrium radius R_{EXPANDED} wherein the sleeve provides at least one of a thin-wall feature and/or an elasticity feature, elasticity properties wherein:
i) according to the thin-wall feature, a ratio between a thickness of the sleeve and the expanded/equilibrium radius R_{EXPANDED} is at most 0.1;
ii) according to the elasticity feature, when the sleeve is deployed within a outer containing rigid tube having a tube radius R_{TUBE} that is 0.8 times the fully-expanded radius R_{EXPANDED}, the sleeve exerts an outward pressure upon the outer containing tube whose value is at least cm of water and at most 200 cm of water;
b) an ETT tube having a proximal and distal end, the ETT longitudinally passing through an interior of the sleeve;
c) a membrane assembly comprising at least one non-membrane that is permanently attached to the sleeve, the at least one membrane being substantially impermeable to liquids, the sleeve, the ETT tube and the membrane assembly being configured such that when the sleeve is deployed within a containing tube so that the sleeve exerts an outward pressure upon a containing tube , the membrane assembly substantially longitudinally seals:
i) an upper region that is outside of tube , within the containing tube and above the contacting non-rigid membrane from
ii) a lower region that is outside of tube within the containing tube , and below the contacting non-rigid membrane .

85. The system of claim 84 wherein the ETT tube has a length between 20 cm and 50 cm.

86. The system of claim 84 wherein the ETT tube includes a collared gas connector at proximal end of ETT tube.

87. The system of claim 84 wherein a distal end of the ETT tube including a Murphy eye and/or a tapered end.
